(19) United States
(12) Patent Application Publication
(10) Pub. No.: US 2023/0002827 A1
(43) Pub. Date: Jan. 5, 2023

(54) METHODS AND COMPOSITIONS FOR THE DETECTION, CLASSIFICATION, AND DIAGNOSIS OF CHRONIC POST-SURGICAL PAIN

(71) Applicant: CHILDREN'S HOSPITAL MEDICAL CENTER, Cincinnati, OH (US)

(72) Inventors: Vidya CHIDAMBARAN, Cincinnati, OH (US); Lisa MARTIN, Cincinnati, OH (US); Lili DING, Cincinnati, OH (US)

(73) Assignee: CHILDREN'S HOSPITAL MEDICAL CENTER, Cincinnati, OH (US)

(21) Appl. No.: 17/432,656

(22) PCT Filed: Feb. 20, 2020
(86) PCT No.: PCT/US2020/019073

§ 371 Date: Aug. 20, 2021

Related U.S. Application Data
(60) Provisional application No. 62/808,384, filed on Feb. 21, 2019.

(51) Int. Cl. C12Q 1/6883; A61K 31/445; C12Q 1/6827; G16H 50/30

(52) U.S. Cl. CPC C12Q 1/6883; A61K 31/445; C12Q 1/6827; G16H 50/30; C12Q 2600/156

(57) ABSTRACT

A method of assessing risk of chronic post-surgical pain (CPSP) in a subject based on genetic factors, non-genetic factors, or a combination thereof. Genetic factors include a genetic profile comprising a combination of single nucleotide polymorphisms (SNPs) in one or more of the specific genes disclosed herein. Non-genetic factors include psychological factors and psychophysical factors. Any of the methods disclosed herein may further comprise applying to the subject a pain management approach based on the subject's risk of developing CPSP.

### CROSS REFERENCE TO RELATED APPLICATIONS

This application claims the benefit of U.S. Provisional Application 62/808,384, filed Feb. 21, 2019, the disclosure of which is hereby incorporated by reference in its entirety.

### BACKGROUND OF THE INVENTION

Chronic postsurgical pain (CPSP) refers to chronic pain that develops or increases in intensity after a surgical procedure and persists beyond the healing process. It is an underrecognized and consequently, undertreated problem, contributing significantly to prolonged opioid use and subsequent misuse. Furthermore, CPSP negatively impacts psychological health and quality of life, leading to disability and suicidal ideation. In order to curb this problem, development of targeted and individualized pain management strategies would be beneficial; however, there are critical gaps in the ability to predict individual risk for CPSP.

Preoperative pain, surgical duration and psychological factors like childhood anxiety sensitivity index (CASI) are associated with CPSP risk in adolescents undergoing spine surgery. However, these factors only explain 16% of variability with medium accuracy in predicting CPSP. Thus, objective biomarkers with higher accuracy are needed to guide preventive and management strategies toward. Incorporation of genetic risk in predicting CPSP could effectively improve predictive accuracy; however, to date only small effect sizes of single variants can explain only a low percentage of the phenotypic variance. Combined effects of a large number of susceptibility loci may become large enough to be useful for targeted risk prediction and prevention. As such, development of a method of assessing polygenic risk profiling for CPSP may have translational potential as a predictive and prognostic biomarker as a PRS is used to model weak contributions of several variants, whereby an individual's genetic risk is the sum of all their risk alleles weighted by significance of the corresponding allele in genome wide association studies.

### SUMMARY OF THE INVENTION

The present disclosure is based, at least in part, on the identification of genetic factors and non-genetic factors as indicators for chronic post-surgical pain (CPSP) in human subjects, either taken alone or in combination. The genetic factors may include single nucleotide polymorphism (SNP) clusters in one or more genes involved in various biological pathways. The non-genetic factors include childhood anxiety sensitivity index (CASI), pain catastrophizing (PCS-C) score, pediatric pain sensitivity score, surgical duration, and area under curve (AUC) of a pain score at days 1 and 2 post-surgery (Pain_AUC_POD12). In some instances, the non-genetic factors may further comprise one or more psychophysical factors of the subject. Accordingly, provided herein are methods and kits for assessing risk of CPSP in a subject relying on the genetic factors, the non-genetic factors, or a combination thereof. Based on the results achieved from the methods disclosed herein, suitable pain management approaches may be designed and applied to the subject.

In one aspect, the present disclosure features a method of assessing risk of chronic post-surgical pain (CPSP) in a subject, the method comprising: (i) obtaining a biological sample from a subject; (ii) analyzing the biological sample to determine a genetic profile of the subject, wherein the genetic profile comprises a combination of single nucleotide polymorphisms (SNPs) in one or more of the genes of ATXN1, PRKCA, CACNG2, CTSG, DRD2, KCND2, KCNJ3, KCNJ6, and KCNK3; (iii) calculating a polygenic risk score (PRS) based on the genetic profile determined in step (ii); and (iv) assessing a risk of developing CPSP in the subject based on the PRS.

In some embodiments, the genetic profile may comprise a combination of SNPs, which comprises SNPs selected from the following: (a) rs61131185, rs12665284, rs493352, rs12198202, rs1150635, rs3812204, rs4716060, rs531089, and/or rs6459476 in ATXN1; (b) rs9754467 in CACNG2; (c) rs1957523 in CTSG; (d) rs7125415 in DRD2; (e) rs202146909 and/or rs1992701 in KCNJ3; (f) rs2850125 in KCNJ6; (g) rs2891519 in KCNK3; (h) rs7809109 in KCND2; and (i) rs62069959, rs11079653, rs9914723, rs7220480, rs200369418, rs227912, and/or rs744214 in PRKCA. For example, the combination of SNPs may comprise of all of (a)-(f). In some examples, the combination of SNPs may consists of all of (a)-(f).

In some embodiments, the genetic profile may comprise a combination of SNPs from one or more genes of CACNG2, DRD2, KCND2, KCNJ3, KCNJ6, and PRKCA. In some examples, the combination of SNPs comprise (a) rs9754467 and/or rs713952 in CACNG2; (b) rs7125415 in DRD2; (c) rs17376373, rs10488301, rs7809109, rs67881942, and/or rs17142908 in KCND2; (d) rs202146909, rs77929576, and/or rs1992701 in KCNJ3; (e) rs7281804, rs2836019, rs2850125, rs1892681, rs11088404, rs858018, rs13048511, rs7280905, and/or rs2032090 in KCNJ6; and/or (f) rs4791072, rs11079653, rs62069959, rs744214, rs7225452, rs6504424, rs973753, rs61687889, rs56399646, rs9635753, rs9914792, rs7220480, rs9914723, rs9903921, rs35105653, rs200369418, rs227912, rs58162388, rs9890911, rs11079656, rs12945884, and/or rs7218425 in PRKCA. In specific examples, the combination of SNPs may comprise (e.g., consist of) all of (a)-(f).

Any of the methods disclosed herein may further comprise determining one or more non-genetic factors of the subject. Examples of the one or more non-genetic factors may be selected from childhood anxiety sensitivity index (CASI), pain catastrophizing (PCS-C) score, pediatric pain sensitivity score, surgical duration, and area under curve (AUC) of a pain score at days 1 and 2 post-surgery (Pain_AUC_POD12). In some instances, the PRS can be weighted by, e.g., regression coefficients of the SNPs in the SNP combination to produce a weighted PRS.

In some embodiments, the risk of CPSP assessed in step (iv) can be based on the weighted PRS in combination with one or more of the non-genetic factors by a regression model. In other embodiments, the risk of CPSP assessed in step (iv) can be based on: (a) CASI, surgical duration, Pain_AUC_POD12, and weighted PRS; or (b) CASI and weighted PRS.

Alternatively or in addition, any of the methods disclosed herein may further comprise applying to the subject a pain management approach based on the subject's risk of developing CPSP. In some embodiments, the pain management approach comprises administration of an analgesic to the subject before and/or after the surgery. Such an analgesic may be locally or systemically administered. Exemplary analgesics include, but are not limited to, bupivacaine, ropivacaine, prilocaine, mepivacaine, chloroprocaine, lidocaine, gabapentin, morphine, fentanyl, oxycodone, hydrocodone, codeine, clonidine, or a combination thereof. In other embodiments, the pain management approach may comprise a psychosocial therapy.

In another aspect, the present disclosure provides a method for determining a genetic profile, the method comprising: (a) obtaining a biological sample from a subject in need thereof, (b) isolating nucleic acids from the biological sample, (c) detecting a combination of single nucleotide polymorphisms (SNPs) in one or more of the genes selected from the group consisting of ATXN1, PRKCA, CACNG2, CTSG, DRD2, KCND2, KCNJ3, KCNJ6, and KCNK3; and (d) determining a genetic profile of the subject based on the SNP combination detected in step (c). The combination of SNPs in the genetic profile may be any of those disclosed herein, e.g., those listed above. In some embodiments, the biological sample from a subject in need thereof can be blood, plasma, salvia, urine, sweat, feces, a buccal smear, or a tissue.

In any of the methods disclosed herein, the subject can be a human patient who is scheduled for or has undergone a surgery. In some examples, the human patient is a child (e.g., younger than 10) or an adolescent (e.g., younger than 19). In some examples, the surgery is a spine and/or pectus surgery, for example, spine fusion.

In yet another aspect, provided herein is a kit for determining a genetic portfolio of a subject, the kit comprising: (i) means for determining a combination of single nucleotide polymorphisms (SNPs) in one or more of the genes selected from ATXN1, PRKCA, CACNG2, CTSG, DRD2, KCND2, KCNJ3, KCNJ6, and KCNK3. The combination of SNPs in the genetic profile may be any of those disclosed herein, e.g., those listed above.

In some embodiments, the means for determining the combination of SNPs comprise a set of primer pairs, each of which is for amplifying a fragment encompassing one of the SNPs in the combination. The set of primer pairs collectively amply fragments encompassing the SNPs in the combination. In other embodiments, the means for determining the combination of SNPs comprise a set of oligonucleotides, each of which is for detecting one of the SNPs in the combination. The set of oligonucleotides collectively detects the SNPs in the combination. In some examples, the set of oligonucleotides is attached to a microarray chip.

In some embodiments, any of the kits disclosed herein may further comprise (ii) a tool for collecting a biological sample from a subject; (iii) a container for placing the biological sample; and/or (iv) one or more reagents for extracting nucleic acids from the biological sample.

Also within the scope of the present disclosure is a method of assessing risk of chronic post-surgical pain (CPSP) in a subject, the method comprising: (i) determining one or more non-genetic factors of a subject; and (ii) assessing risk of CPSP of the subject based on the one or more non-genetic factors. The non-genetic factors include one or more of childhood anxiety sensitivity index (CASI), pain catastrophizing (PCS-C) score, pediatric pain sensitivity score, surgical duration, and area under curve (AUC) of a pain score at days 1 and 2 post-surgery (Pain_AUC_POD12). In some embodiments, the method may further comprise applying to the subject a pain management approach based on the subject's risk of developing CPSP, for example, those disclosed herein. The subject may be a human patient who is scheduled for or has undergone a surgery. In some instances, the human patient is a child or an adolescent. In some instances, the surgery is a spine and/or pectus surgery, for example, spine fusion.

In addition, the present disclosure provides use of any of the pain management approaches for managing CPSP in a subject in need thereof, wherein the subject is at risk for CPSP as determined by any of the methods disclosed herein.

The details of one or more embodiments of the invention are set forth in the description below. Other features or advantages of the present invention will be apparent from the following drawings and detailed description of several embodiments, and also from the appended claims.

### BRIEF DESCRIPTION OF THE DRAWINGS

The following drawings form part of the present specification and are included to further demonstrate certain aspects of the present disclosure, which can be better understood by reference to the drawing in combination with the detailed description of specific embodiments presented herein.

FIG. 1 is a graph showing that the fit of AUC with CPSP (NRS> 3/10 at 12 months) was significantly higher AUC in CPSP subjects compared to no CPSP (p<0.0001).

FIG. 2 is a graph showing linear pain trajectories (fitted linear regression lines) and their 95% prediction intervals (shaded bands) of four combinatorial patient cohorts with and without CPSP are plotted. 66% of patients had clear pain trajectories (either low or high pain scores from preoperative to years later).

FIG. 3 is a graph showing the receiver operating characteristic (ROC) for psychosocial factor model prediction of CPSP where AUC is 75%

FIG. 4 is a graph showing 5 phenotype clusters that were identified based on acute postoperative pain severity (PoP), CPSP (PP) and CASI. For example, cluster 1 has 100% of pts with low CASI, ˜80% of the same pts have high PoP, and ˜40% the same pts have high risk of PP. All groups have equal distribution of subjects. Group 4 has high risk for all factors while group 3 is protective; other subgroups have different risk combinations, implying varying strategies will be required per subgroup.

FIG. 5 is a graph showing a visual representation of co-clustering of PRS developed for CPSP, acute pain and CASI with the 5 phenotype clusters, suggests genotype underpinnings for each phenotype group is different and can be identified.

FIGS. 6A-6E include graphs showing preoperative psychophysical testing in seven patients undergoing pectus surgery. FIG. 6A: shows results of temporal summation (TS) testing before surgery in the patients. FIG. 6B: shows results of pressure pain threshold (PTT) testing before surgery in the patients. FIG. 6C: shows 2 point discrimination in the patients. FIG. 6D: shows results of cold pain unpleasantness and intensity 20 seconds after submersion in ice water before surgery in the patients. FIG. 6E: shows results of conditioned pain modulation (CPM) testing before surgery in the patients.

FIG. 7 is a graph showing pain trajectories based on average pain scores over different time points for each patient after pectus surgery. AUC is defined as the area under the curve of the trajectory.

FIG. 8 is a diagram showing preoperative sensory testing profiles per pectus subject (upper panel) and corresponding AUC (pain cumulative experience) shows feasibility of QST and differences as anticipated (lower panel).

FIG. 9 is an image of phenotype clusters of patients who underwent pectus surgery where the phenotype clusters indicate difference in clustering are likely defined by few sensory responses, namely CPM

FIG. 10 is a diagram showing enrichment for significant associations with CPSP in the genetic data obtained from subjects within the Scoliosis Cohort. The number of SNPs in the set compared to 10000 control runs (0=training set; 1-10 deciles of test sets) is significant for training set (table to the right) indicated by red dot (p<0.05) in the graph to the left.

FIG. 11 is a graph showing the predicted probability (with 95% CT) of CPSP for a subject having a median (for the cohort) CASI=28.16 using the regression model plotted as a function of the PRS.

FIG. 12 is a graph showing the ROC for risk model with PRS, CASI, surgical duration and preoperative pain predicts CPSP with 92% accuracy (AUC=0.92).

FIG. 13 is an image showing a risk decision tree for CPSP based on the 80 SNPs associated with CPSP and calculated with the J48 Algorithm (Partykit Graphics).

FIG. 14 is an image showing the workflow protocol for identification of polygenic risk scores for CPSP.

FIG. 15 is a graph showing the ROC that showed the sensitivity/1-specificity for prediction of chronic post-surgical pain using the non-genetic model (including childhood anxiety sensitivity index (CASI) surgical duration and acute postoperative pain) compared with the prediction using the polygenic risk score final model (PRS and CASI). The area under curve for genetic model is 0.97 (95% CI 0.93-0.99) compared to 0.77 (95% CI 0.66-0.87) for non-genetic model (p=0.0007).

FIG. 16 is an image showing an exemplary decision tree using simple logical rules shows that with just 3 variants, the risk for chronic postsurgical pain was classified with reasonable accuracy. At each node, counts for high vs. low risk group were included and edges were defined by the genotypes used. Objects with solid outline indicate high risk and objects with dashed outline indicate low risk, respectively. PRKCA: Protein Kinase C Alpha; ATXN1: Ataxin 2

### DETAILED DESCRIPTION OF THE INVENTION

The present disclosure is based, at least in part, on the discovery of genetic and non-genetic factors, which, either taken alone or in combination, can be relied on to predict chronic postsurgical pain (CPSP) in a subject, such as a child or an adolescent. The genetic factors include single nucleotide polymorphisms (SNPs) in specific genes. The non-genetic factors include, for example, childhood anxiety sensitivity index (CASI), pain catastrophizing (PCS-C) score, pediatric pain sensitivity score, surgical duration, and area under curve (AUC) of a pain score at days 1 and 2 post-surgery (Pain_AUC_POD12).

Accordingly, provided herein are methods for assessing CPSP risk in a subject relying on genetic factors, non-genetic factors, or a combination thereof. Such methods support for CPSP risk stratification and development of tailored prevention. For example, the assessment obtained from such methods would facilitate selecting suitable pain management approaches and applying such to the subject.

### I. Chronic Postsurgical Pain (CPSP) Assessment

In some aspects, provided herein are methods for assessing risks for CPSP in a subject based on any of the genetic factors, non-genetic factors, or a combination thereof as biomarkers. Such methods may comprise (i) analyzing a subject's genetic profile comprising any of the SNP combinations disclosed herein, a non-genetic profile comprising any combination of the non-genetic factors also disclosed herein for, or a combination thereof, (ii) calculating a polygenic risk score (PRS), which may be based on the genetic profile alone or in combination with the non-genetic profile, and (iii) assessing CPSP risk in that subject. Alternatively, the method may comprise analyzing a subject's non-genetic profile as disclosed herein an assessing CPSP risk for that subject. Bases on the CPSP risk assessed by any of the methods disclosed herein, a tailored pain management approach can be developed and applied to that subject.

(A) Genetic Factors

A computational biology approach may be applied to identify SNPs associated with the risk of CPSP. For example, ranked prioritized variant sets can be tested for their association with CPSP against a plurality of matched control gene set. Covariates can be adjusted to identify a set of variants enriched for CPSP (e.g., p<0.001). A regression model such as the LASSO regression model may be applied to the enriched variants set to further select SNPs that are relevant to CPSP risk.

In some embodiments, a genetic profile for CPSP risk assessment may comprise a combination of SNPs selected from those listed in Table 6 below. As used herein, “a combination of SNPs” refers to two or more SNPs. Such a SNP combination may contains at least 5 SNPs, at least 10 SNPs, at least 15 SNPs, at least 20 SNPs, at least 25 SNPs, at least 30 SNPs, at least 35 SNPs, at least 40 SNPs, at least 45 SNPs, at least 50 SNPs, at least 55 SNPs, at least 60 SNPs, at least 65 SNPs, at least 70 SNPs, or at least 75 SNPs selected from Table 6.

In some embodiments, a genetic profile for CPSP risk assessment may comprise a combination of SNPs in one or more of the genes of ATXN1; CACNG2; CTSG; DRD2; HLA-DQB1; IL10; KCNA1: KC ND2; KCNJ3; KCN16; KCNK3; and PRKCA. Exemplary biological pathways that these genes are involved are provided in Table 8 below. In some embodiments, the genetic profile comprises a combination of SNPs in one or more of the genes of ATXN1, PRKCA, CACNG2, CTSG, DRD2, KCND2, KCNJ3, KCNJ6, and KCNK3. In other embodiments, the genetic profile comprises a combination of SNPs in one or more of the genes CACNG2, DRD2, KCND2, KCNJ3, KCNJ6, and PRKCA.

In some embodiments, the SNP combination as disclosed herein may be selected from:
(a) rs61131185, rs12665284, rs493352, rs12198202, rs1150635, rs3812204, rs4716060, rs531089, and/or rs6459476 in ATXN1;
(b) rs9754467 and/or rs713952 in CACNG2;
(c) rs1957523 in CTSG;
(d) rs7125415 in DRD2;
(e) rs202146909, rs77929576, and/or rs1992701 in KCNJ3;
(f) rs7281804, rs2836019, rs2850125, rs1892681, rs11088404, rs858018, rs13048511, rs7280905, and/or rs2032090 in KCNJ6;
(g) rs2891519 in KCNK3;
(h) rs17376373, rs10488301, rs7809109 rs67881942, and/or rs17142908 in KCND2; and
(i) rs4791072, rs11079653, rs62069959, rs744214, rs7225452, rs6504424, rs973753, rs61687889, rs56399646, rs9635753, rs9914792, rs7220480, rs9914723, rs9903921, rs35105653, rs200369418, rs227912, rs58162388, rs9890911, rs11079656, rs12945884, and/or rs7218425 in PRKCA.

In some examples, the genetic profile disclosed herein comprises a SNP combination that comprise at least one SNP in each of (a)-(i). Alternatively or in addition, the SNP combination contains at least 5 SNPs, at least 10 SNPs, at least 15 SNPs, at least 20 SNPs, at least 25 SNPs, at least 30 SNPs, at least 35 SNPs, at least 40 SNPs, at least 45 SNPs or at least 50 SNPs selected from (a)-(i). In some instances, the SNP combination comprises PRKCA rs9914723 and PRKCA rs62069959. In other examples, the SNP combination may comprise or consists of rs9914723 and rs62069959 in PRKCA, and rs493352 in ATXN1. The correlation between the various alleles of these SNPs and the high or low risk for CPSP can be found in FIG. 16.

In specific examples, the SNP combination may comprise or consists of 24 SNPs, including: (a) rs61131185, rs12665284, rs493352, rs12198202, rs1150635, rs3812204, rs4716060, rs531089, and rs6459476 in ATXN1; (b) rs9754467 in CACNG2; (c) rs1957523 in CTSG; (d) rs7125415 in DRD2; (e) rs202146909 and rs1992701 in KCNJ3; (0 rs2850125 in KCNJ6; (g) rs2891519 in KCNK3; (h) rs7809109 in KCND2; and (i) rs62069959, rs11079653, rs9914723, rs7220480, rs200369418, rs227912, and rs744214 in PRKCA.

In other examples, the SNP combination may comprise or consists of (a) rs9754467 and/or rs713952 in CACNG2; (b) rs7125415 in DRD2; (c) rs17376373, rs10488301, rs7809109, rs67881942, and rs17142908 in KCND2; (d) rs202146909, rs77929576, and rs1992701 in KCNJ3; (e) rs7281804, rs2836019, rs2850125, rs1892681, rs11088404, rs858018, rs13048511, rs7280905, and rs2032090 in KCNJ6; and (f) rs4791072, rs11079653, rs62069959, rs744214, rs7225452, rs6504424, rs973753, rs61687889, rs56399646, rs9635753, rs9914792, rs7220480, rs9914723, rs9903921, rs35105653, rs200369418, rs227912, rs58162388, rs9890911, rs11079656, rs12945884, and rs7218425 in PRKCA.

Any of the SNP combinations disclosed herein may be determined by conventional methods using a biological sample obtained from a target subject. The biological sample may be of any type that contains genetic materials. Examples include blood, plasma, salvia, urine, sweat, feces, a buccal smear, or a tissue. A materials can be extracted from the biological samples and subject to analysis as known in the art to determine genotype of the SNP combination as disclosed herein.

In some embodiments, analysis of the SNPs can be carried out by amplification of the region encompassing a particular target SNP according to amplification protocols well known in the art (e.g., polymerase chain reaction, ligase chain reaction, strand displacement amplification, transcription-based amplification, self-sustained sequence replication (3SR), Q-Beta replicase protocols, nucleic acid sequence-based amplification (NASBA), repair chain reaction (RCR) and boomerang DNA amplification (BDA)). The amplification product can then be visualized directly in a gel by staining or the product can be detected by hybridization with a detectable probe. When amplification conditions allow for amplification of all allelic types of a biomarker, the types can be distinguished by a variety of well-known methods, such as hybridization with an allele-specific probe, secondary amplification with allele-specific primers, by restriction endonuclease digestion, or by electrophoresis.

Thus, in some examples, determining each SNP in a SNP combination as disclosed herein can encompass a set of primer pairs, each of which is for amplifying a fragment encompassing one of the SNPs in the combination. The set of primer pairs collectively amply fragments encompassing the SNPs in the combination.

In other embodiments, SNPs in a SNP combination disclosed herein may be determined by microarray assays, which are also well known in the art. In some instances, determining a combination of SNPs disclosed herein can encompass a set of oligonucleotides, each of which is for detecting one of the SNPs in the combination. The set of oligonucleotides collectively detects the SNPs in the combination. The oligonucleotide for detecting a target SNP may comprise one probe that is differentially hybridizable to one allele of the SNP. Alternatively, the oligonucleotide for detecting a target SNP may comprise a pair of probes, one of which is differentially hybridizable to one allele and the other of which is differentially hybridizable to the other allele. In some embodiments, a set of oligonucleotides can be 5′ and 3′ oligonucleotides flanking a SNP site of interest. In some embodiments, for every SNP, at least 2 to 8 oligonucleotides (2 complimentary pairs) can be synthesized, at least one pair for each allele.

The set of oligonucleotides for detecting the SNP combination may be immobilized on a support member to form a gene chip. Gene chips, also called “biochips” or “arrays” or “microarrays” are miniaturized devices typically with dimensions in the micrometer to millimeter range for performing chemical and biochemical reactions and are suited for performing the methods disclosed herein. Arrays may be constructed via microelectronic and/or microfabrication using essentially any and all techniques known and available in the semiconductor industry and/or in the biochemistry industry, provided that such techniques are amenable to and compatible with the deposition and screening of polynucleotide sequences. Microarrays are particularly desirable for their virtues of high sample throughput and low cost for generating profiles and other data.

Any of the methods for determining a genetic profile comprising any of the SNP combinations disclosed herein is also within the scope of the present disclosure.

(B) Non-Genetic Factors

In some embodiments, non-genetic factors associated with CPSP can be one or more non-genetic factors such as psychosocial factors, one or more psychophysical factors, or a combination thereof. Exemplary psychosocial factors include, but are not limited to, anxiety, depression, somatization, stress, cognition, and pain perception. Exemplary psychophysical factors include, but are not limited to, pressure pain threshold (PPT), conditioned pain modulation (CPM), and temporal summation (TS).

Psychosocial factors associated with a subject may be determined by testing the subject with at least one psychosocial questionnaire comprising one or more questions for assessing the one or more psychosocial factors noted above. Examples of such questionnaire include, but are not limited to, Eysenck Personality Questionnaire, Life Experiences Survey, Perceived Stress Scale, State-Trait Anxiety Inventory (STAI) Form Y-2, STAI Form Y-1, Pittsburgh Sleep Quality Index, Kohn Reactivity Scale, Pennebaker Inventory for Limbic Languidness, Short Form 12 Health Survey v2, SF-36, Pain Catastrophizing Scale, In vivo Coping Questionnaire, Coping Strategies Questionnaire-Rev, Lifetime Stressor List & Post-Traumatic Stress Disorder (PTSTD) Checklist for Civilians, Multidimensional Pain Inventory v3, Comprehensive Pain & Symptom Questionnaire, Symptom Checklist-90-R(SCL-90R), Brief Symptom Inventory (BSI), Beck Depression Inventory (BDI), Profile of Mood States Bi-polar, Pain Intensity Measures, Pain Unpleasantness Measures, childhood anxiety sensitivity index (CASI), pain catastrophizing (PCS-C) score, Pediatric Pain Screening Tool, NIH Patient-Reported Outcome Measurement Information System (PROMIS) Pediatric Short Form v1.1, pediatric pain sensitivity score, PCS-Parent (PCS-P), Electronic pain diary/PainDETECT (PD), Adult Responses to Children's Symptoms (ARCS), Functional disability Index (FDI), Pediatric Quality of Life Inventory 4.0 Generic Core Scales (PedsQL), surgical duration, and area under curve (AUC) of a pain score at days 1 and 2 post-surgery (Pain_AUC_POD12).

Any of the psychophysical factors of a subject, e.g., PPT, CPM, and TS, can be assessed by conventional practice. PPT refers to the minimum force applied to a subject that induces pain. See, e.g., Park et al., Ann Rehabil Med. 2011, 35(3):412-417. Conditioned pain modulation (CPM) is a psychophysical experimental measure of the endogenous pain inhibitory pathway in humans. Kennedy et al., Pain 2016, 157(11):2410-2419. Temporal summation is a clinical measure of central sensitization in which a high frequency of action potentials in the presynaptic neuron elicits postsynaptic potentials that overlap and summate with each other.

In some examples, one of more of the following non-genetic factors may be determined in a subject for assessing the subject's risk for CPSP, either taken alone or in combination with any of the genetic factors disclosed herein: childhood anxiety sensitivity index (CASI), pain catastrophizing (PCS-C) score, pediatric pain sensitivity score, surgical duration, and area under curve (AUC) of a pain score at days 1 and 2 post-surgery (Pain_AUC_POD12).

CASI typically measures anxiety sensitivity, which is an established cognitive risk factor for anxiety disorders. In some examples, CASI may be comprised of lower-order factors pertaining to physical, psychological and social concerns. Factor structure of CASI is well known in the art. See, e.g., Silverman et al., Behav, Res. Ther, 1999, 37(9):903-917; and Muris, Behav, Res. Ther. 2002, 40(3):299-311.

The PCS-C score of a subject influences that subject's adjustment to pain. In some instances, PCS-C scores can be determined by testing a subject (e.g., children or parents) with questionnaires assessing catastrophizing about pain in the subject. Child versions of PCS-C and parent versions of PCS-C are known in the art. See, for example, Crombez et al., Pain 2003 104(3):639-46 and Goubert et al., Pain 2006 123(3):254-263.

Measurement of other non-genetic factors disclosed herein is also known in the art. See, e.g., Simons et al., Pain, 2015, 156(8):1511-1518.

(C) Calculation of Polygenic Risk Scores (PRS) and CPSP Risk Assessment

In some embodiments, a polygenic risk score (PRS) can be calculated based on the genetic profile of a subject as disclosed herein, optionally in combination with one or more non-genetic factors as also disclosed herein. A PRS for a subject may be calculated using a computational method as known in the art based on the SNP status of those SNPs in a genetic profile of a subject. For example, logistic and/or linear models may be used for PRS calculation. In other examples, a multiple regression model may be used.

In some examples, the PRS may be calculated as a weighted sum of products between number of risk alleles and their corresponding regression coefficients. Risk alleles of exemplary SNPs as disclosed herein and their exemplary regression coefficients are provided in Table 6. In some examples, SNPs provided in Table 9 can be used for PRS calculation. In some examples, bootstrapping may be applied to validate the prediction model. For example, PRS may be weighted by regression coefficients of the SNPs in the SNP combination to produce a weighted PRS.

In some embodiments, a PRS of a subject can be used to predict risk of CPSP for that subject. The PRS of a candidate subject (e.g., a human subject) may be compared with a reference value. In some examples, the reference value may represent a PRS of a subject of the same species as the candidate subject (e.g., a human subject) and having CPSP, wherein the PRS is calculated by the same method (e.g., same computational model) based on the same SNP combination. In other examples, the reference value may represent a PRS of a subject of the same species as the candidate subject (e.g., a human subject) and having no CPSP, wherein the PRS is calculated by the same method (e.g., same computational model) based on the same SNP combination. Such reference values in association with a particular SNP combination can be predetermined based on PRS scores of subjects representing high CPSP risk or minor CPSP risk. By comparing the PRS of a candidate subject with a corresponding reference value (e.g., PRS calculated by the same method based on the same SNP combination), the risk for CPSP for the candidate subject can be determined. For example, if the PRS of a candidate subject is close to a corresponding reference value representing high CPSP risk, that candidate subject can be predicted as having a high risk for CPSP. Alternatively, if the PRS of a candidate subject is close to a corresponding reference value representing minor CPSP risk, that candidate subject can be predicted as having a low risk for CPSP.

In some embodiments, the CPSP risk for a subject may be assessed by a PRS of that subject alone. In other embodiments, at least one non-genetic factor, for example, a psychosocial factor, a psychophysical factor, or a combination thereof, can be used as a covariate in combination with a PRS for assessment of CPSP risk. Such non-genetic factors may be used in a multiple regression model to assess risk for CPSP.

In some examples, a full regression model may be applied in assessing risk for CPSP, taking into consideration weighted PRS in combination with multiple non-genetic factors, for example, CASI, surgical duration, and Pain_AUC_POD12. In other examples a reduced regression model may be used for assessing risk for CPSP, taking into consideration weighted PRS in combination with one non-genetic factor, for example, CASI. See also Table 10.

Alternatively or in addition, the risk for CPSP can be assessed by one of the genetic factors as disclosed herein in combination with one or more non-genetic factors. In some examples, the non-genetic factors may comprise one or more of psychosocial factors as those disclosed herein. In other examples, the non-genetic factors may comprise one or more psychophysical factors such as those disclosed herein. In yet other examples, the non-genetic factors may comprise at least one psychosocial factor and at least one psychophysical factor.

Any of the CPSP risk assessment methods disclosed herein may be applied to a subject, who can be a human subject or a non-human mammal. In some embodiments, the subject is a human subject, for example a human child or a human adolescent. An adolescent can be any human between ages of about 10 years old to about 19 years old. A child can be any human under the age of about 10 years old. In some embodiments, the human subject is a young person. A young person can be any human between the aged of about 10 years old to about 24 years old. In some embodiments, the human subject is an adult. An adult can be any human older than about 19 years of age.

In some embodiments, the subject is a human patient (e.g., a child or an adolescent) who is scheduled for a surgery. In other embodiments, the subject is a human patient (e.g., a child or an adolescent) who or has undergone a surgery. In this case, the assessment method disclosed herein may be applied to that subject within a suitable period after the surgery, for example, within 7 days, within 5 days, within 3 days, or within 2 days. In some specific examples, the assessment method may be applied to the subject within 48 hours or with 24 hours after the surgery.

Non-limiting examples of surgical procedures include amputation, appendectomy, carotid endarterectomy, cataract surgery, cesarean section, cholecystectomy, coronary artery bypass, craniotomy, dental surgery hip arthroplasty, sternotomy, thoracotomy, vasectomy, melanoma resection, hysterectomy, inguinal hernia repair, low back pain surgery, mastectomy, colectomy, prostatectomy, tonsillectomy, and orthopedic surgery. In some embodiments the surgery is a spine and/or pectus surgery. In some examples, the surgery is idiopathic scoliosis, pectus excavatum, and/or kyphosis undergoing posterior spine fusion.

In some embodiments the subject is not a female who were pregnant or breastfeeding. In some embodiments, the subject is not a human patient who has been diagnosed as having chronic pain. In other embodiments, the subject is free of any opioid in the past six months before the assessment. Alternatively or in addition, the subject is not a human patient having hepatic and/or renal disease or having developmental delays.

### II. Tailored Pain Management Based on CPSP Risk Assessment

After the risk for CPSP of a subject has been assessed using any of the methods disclosed herein, a tailored pain management approach may be determined based on that subject's risk for CPSP. As such, any of the methods disclosed herein may further comprise applying to the subject a pain management approach based on the subject's risk of developing CPSP.

A pain management approach may comprise the use of an analgesic, application of a one psychosocial therapy, or a combination thereof. Examples of analgesics include, but are not limited to bupivacaine, ropivacaine, prilocaine, mepivacaine, chloroprocaine, lidocaine, gabapentin, morphine, fentanyl, oxycodone, hydrocodone, codeine, and clonidine. In some examples, the analgesic may be systemically administered. In other examples, the analgesic may be locally applied, e.g., to the surgery site. Non-limiting examples of psychosocial therapy include psychotherapy, psychoeducation, self-help, support groups, psychosocial rehabilitation, contingency management, cognitive behavioral therapy, and assertive community treatment.

In some embodiments, a pain management approach based on the subject's risk of developing CPSP may include administering a physician-guided opioid medication management and tapering regimen, an opioid-sparing pharmacotherapy, a non-opioid pharmacotherapy, and/or a combination thereof after surgery to prevent CPSP. In some embodiments, a physician-guided opioid medication tapering regimen concludes about 2 to about 5 months after surgery. In some embodiments, a physician-guided opioid medication tapering regimen concludes about 3 months after surgery.

A suitable pain management approach may be selected based on the subject's risk for CPSP. For example, if a subject is assessed as having low risk for CPSP, one or more psychosocial therapies may be used, either taken alone or in combination with a mild analgesic. On the other hand, if a subject is assessed as having a high risk for CPSP, a strong analgesic may be selected, either taken alone or in combination with one or more psychosocial therapies. Choosing suitable pain management approaches for subjects having different levels of CPSP risk would be within the knowledge of medical practitioners.

### III. Kit for CPSP Risk Assessment

In another aspect, the present disclosure provides a kit for determining a genetic profile and optionally for assessing the risk for CPSP. Such a kit may comprise means for determining any of the SNP combinations disclosed herein, for example, means for determining the 24 SNPs listed in Table 9.

In some embodiments, the means for determining genetic status of the SNP combination may comprise a set of primer pairs. Each of the primer pairs are designed for amplifying a fragment (e.g., around 150 bp or around 100 bp) encompassing the site of a target SNP. The set of primer pairs, collectively, are designed for amplifying fragments encompassing the sites of all target SNPs in a SNP combination, e.g., those disclosed herein.

In other embodiments, the means for determining genetic status of the SNP combination may comprise a set of oligosaccharides. Each of the oligonucleotides is designed for detecting a target SNP in the combination and the whole set, collectively, is designed for detecting all SNPs in the combination. Such an oligonucleotide may be differentially hybridizable to one allele of a SNP (e.g., hybridizable to only allele under certain hybridization conditions). Design of such an oligonucleotide for detecting a particular allele of a SNP is within the knowledge of a skilled person in the art. See, e.g., Sambrook et al. et al., MOLECULAR CLONING—A LABORATORY MANUAL (2ND ED.), Vols. 1-3, Cold Spring Harbor Laboratory, Cold Spring Harbor, N.Y. (1989).

In some example, the kit disclosed herein may comprise a microarray chip comprising a support member, on which the set of oligonucleotides (probes) can be immobilized. The probes may comprise DNA sequences, RNA sequences, or a hybrid of DNA and RNA sequences. The probes may also comprise modified nucleotide residues. The support member in the microarray chip may be either porous or non-porous. For example, the probes may be attached to a nitrocellulose or nylon membrane or filter covalently at either the 3′ or the 5′ end of the polynucleotide. Alternatively, the support member may have a glass or plastic surface. In one embodiment, hybridization levels are measured to microarrays of probes consisting of a solid phase on the surface of which are immobilized a population of polynucleotides, such as a population of DNA or DNA mimics, or, alternatively, a population of RNA or RNA mimics. The solid phase may be a nonporous or, optionally, a porous material such as a gel.

In one embodiment, a microarray chip may comprise a support member with an ordered array of binding (e.g., hybridization) sites or “probes” each representing one of the target SNP described herein. Preferably the microarrays are addressable arrays, and more preferably positionally addressable arrays. For example, each probe of the array is preferably located at a known, predetermined position on the solid support such that the identity (i.e., the sequence) of each probe can be determined from its position in the array (i.e., on the support or surface). In preferred embodiments, each probe is covalently attached to the solid support at a single site.

The microarray chips disclosed herein can be made in a number of ways. In some examples, the microarray chips are reproducible, allowing multiple copies of a given array to be produced and easily compared with each other. In some examples, microarrays are made from materials that are stable under binding (e.g., nucleic acid hybridization) conditions. The microarrays may be small, e.g., between 1 cm² and 25 cm², between 12 cm² and 13 cm², or about 3 cm². However, larger arrays are also contemplated.

Any of the kits disclosed herein may further comprise a container for placing a biological sample, and optionally a tool for collecting a biological sample from a subject. Alternatively or in addition, the kit may further comprise one or more reagents for extracting nucleic acids from the biological sample. In some examples, the kit may comprise reagents for PCR amplification of fragments encompassing target SNP sites. In other examples, the kit may comprise reagents for hybridization.

Any of the kits may further comprise an instruction manual providing guidance for using the kit to determine a genetic profile comprising a combination of the target SNPs as disclosed herein.

In some embodiments, the kit may further comprise questionnaires for assessing one or more of the non-genetic factors associated with the CPSP risk, e.g., CASI, PCS-C, etc. Instructions of how to use such questionnaires for assessing the non-genetic factors may also be included.

Further, any of the kits disclosed herein may comprise a processor, e.g., a computational processor, for PSR calculation and/or CPSP risk assessment. Such a processor may be configured with a regression model such as those disclosed herein. By inputting the genetic profile (genetic status of a SNP combination associated with CPSP) and optionally any of the non-genetic factors, the processor may process the information to predict risk of CPSP.

### General Techniques

The practice of the present disclosure will employ, unless otherwise indicated, conventional techniques of molecular biology (including recombinant techniques), microbiology, cell biology, biochemistry, and immunology, which are within the skill of the art. Such techniques are explained fully in the literature, such as Molecular Cloning: A Laboratory Manual, second edition (Sambrook, et al., 1989) Cold Spring Harbor Press; Oligonucleotide Synthesis (M. J. Gait, ed. 1984); Methods in Molecular Biology, Humana Press; Cell Biology: A Laboratory Notebook (J. E. Cellis, ed., 1989) Academic Press; Animal Cell Culture (R. I. Freshney, ed. 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds. 1993-8) J. Wiley and Sons; Methods in Enzymology (Academic Press, Inc.); Handbook of Experimental Immunology (D. M. Weir and C. C. Blackwell, eds.): Gene Transfer Vectors for Mammalian Cells (J. M. Miller and M. P. Calos, eds., 1987); Current Protocols in Molecular Biology (F. M. Ausubel, et al. eds. 1987); PCR: The Polymerase Chain Reaction, (Mullis, et al., eds. 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practice approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds. Harwood Academic Publishers, 1995); DNA Cloning: A practical Approach, Volumes I and II (D. N. Glover ed. 1985); Nucleic Acid Hybridization (B. D. Hames & S. J. Higgins eds. (1985»; Transcription and Translation (B. D. Hames & S. J. Higgins, eds. (1984»; Animal Cell Culture (R. I. Freshney, ed. (1986»; Immobilized Cells and Enzymes (IRL Press, (1986»; and B. Perbal, A practical Guide To Molecular Cloning (1984); F. M. Ausubel et al. (eds.).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit, and scope of the present disclosure. All such modifications are intended to be within the scope of the disclosure.

### Example 1. Recruitment and Selection for Observational Prospective Cohort

An observational prospective cohort study was conducted in 171 adolescents with idiopathic scoliosis, pectus excavatum, and/or kyphosis undergoing posterior spine fusion using standard surgical techniques, anesthetic and pain protocols. The studies were registered with ClinicalTrials.gov (Identifier: NCT01839461, NCT01731873), the disclosures of which are incorporated herein in their entirety.

Regarding inclusion criteria, healthy non-obese children aged 10-18 years of American Society of Anesthesiologists (ASA) physical status less than or equal to two (mild systemic disease) with a diagnosis of idiopathic scoliosis and/or kyphosis, scheduled to undergo elective spinal fusion were selected for the study. The ASA Physical Status Classification System that was used for inclusion criteria is provided in Table 1 below.

TABLE 1
Physical Status (PS) Classification Levels, Definitions and ASA-Approved
Examples

| ASA PS | | |
|---|---|---|
| Classification | Definition | Examples, Including, but not Limited to: |
| ASA I | A normal healthy patient | Healthy, non-smoking, no or minimal |
| | | alcohol use |
| ASA II | A patient with mild systemic disease | Mild diseases only without substantive |
| | | functional limitations. Examples include |
| | | (but not limited to): current smoker, |
| | | social alcohol drinker, pregnancy, |
| | | obesity (30 < BMI < 40), well-controlled |
| | | DM/HTN, mild lung disease |
| ASA III | A patient with severe systemic disease | Substantive functional limitations; One |
| | | or more moderate to severe diseases. |
| | | Examples include (but not limited to): |
| | | poorly controlled DM or HTN, COPD, |
| | | morbid obesity (BMI ≥ 40), active |
| | | hepatitis, alcohol dependence or abuse, |
| | | implanted pacemaker, moderate |
| | | reduction of ejection fraction, ESRD |
| | | undergoing regularly scheduled dialysis, |
| | | premature infant PCA <60 weeks, |
| | | history (>3 months) of MI, CVA, TIA, |
| | | or CAD/stents. |
| ASA IV | A patient with severe systemic disease | Examples include (but not limited to): |
| | that is a constant threat to life | recent (<3 months) MI, CVA, TIA, or |
| | | CAD/stents, ongoing cardiac ischemia or |
| | | severe valve dysfunction, severe |
| | | reduction of ejection fraction, sepsis, |
| | | DIC, ARD or ESRD not undergoing |
| | | regularly scheduled dialysis |
| ASA V | A moribund patient who is not expected | Examples include (but not limited to): |
| | to survive without the operation | ruptured abdominal/thoracic aneurysm, |
| | | massive trauma, intracranial bleed with |
| | | mass effect, ischemic bowel in the face |
| | | of significant cardiac pathology or |
| | | multiple organ/system dysfunction |
| ASA VI | A declared brain-dead patient whose | |
| | organs are being removed for donor | |
| | purposes | |

Excluded from the study were females who were pregnant or breastfeeding, subjects with a diagnosis of chronic pain or opioid use in the past six months, hepatic/renal disease and/or developmental delays.

The rationale for using spine pediatric cohorts in the study was that idiopathic scoliosis (increased spine curvature), kyphosis (outward spine curvature), and pectus excavatum (caved-in chest) are common, musculoskeletal chest wall deformities. Spine fusion for scoliosis/kyphosis and endoscopic Nuss procedure for pectus were known to be the most painful corrective surgeries healthy children/adolescents undergo. Further, unlike adults, it was known that children/adolescents have minimal/no pain before surgery and surgery was usually the child's first exposure to severe pain and opioids. A 37-40% incidence of CPSP was observed at 2-3 months and 1 year after spine fusion, which aligned with postsurgical outcomes data from international pediatric spine surgery registries.

### Example 2. Assessment of Psychosocial and Perioperative Factors as Predictors of CPSP in a Scoliosis Cohort

A cohort of 171 children undergoing spinal fusion under standard anesthesia/pain protocols was selected according to the protocol in Example 1. Preoperative data on demographics (sex, age, race, etc.), weight, home medications, and scoliosis curve from X-ray reports, were collected from the cohort. The numerical rating scale (NRS) for self-report of pain intensity in children and adolescents—a validated measure of pain in children aged ˜7 to 17 years—was determined in a manner similar to that described in von Baeyer, (2009), European Journal of Pain, 13: 1005-1007, the disclosure of which is incorporated herein in its entirety. Because socioeconomic status can influence pain responses, preoperative data also includes socioeconomic status (SES) data on education level and financial condition of the family. Questionnaires to assess pain catastrophizing (Pain catastrophizing scale/PCS), functional disability (FDI), and anxiety sensitivity (childhood anxiety sensitivity index (CASI)), were administered preoperatively in a manner as described in Crombez et al., (2003) Pain, 104(3):639-646, Walker et al, (1991) Journal of Pediatric Psychology 16(1):39-58, and Silverman et al., (1991) J Clin Child Psychol 20:162-168, respectively, the disclosures of which are incorporated herein in their entirety. In brief, the data for psychosocial variables (e.g., PCS, CASI, FDI), were collected from answers obtained from child and parent questionnaires, the details of which are provided in Table 2 below.

TABLE 2
Validated Questionnaires for Psychosocial Variables Used in Study

| | |
|---|---|
| Questionnaires | Description |

CHILD FACTORS

| | |
|---|---|
| Child Anxiety Sensitivity | 18-item self-report tool to measure anxiety in children and adolescents |
| Index/CASI | (scores: 18-54) with demonstrated high internal consistency, good |
| | test-retest reliability and construct validity. |
| Pediatric Pain Screening | A composite self-report tool evaluating depression, anxiety, pain |
| Tool | catastrophizing and sleep problems validated in chronic pediatric pain; Has |
| | physical and psychosocial subscales. |
| NIH Patient-Reported | The PROMIS Depression 8a, is an 8-item short form which assesses self- |
| Outcome Measurement | reported negative mood (sadness), views of self (worthlessness), social |
| Information System | cognition, and decreased positive affect. It is validated in 8-17 year olds. |
| (PROMIS) Pediatric | Data will also be collected using NIH PROMIS 8-item Pediatric Pain |
| Short Form v1.1 | Interference, 10-item Fatigue, 8-item Pain Behavior Scales, for future |
| | evaluation. |
| Coping with pain | 39-item self-report assesses pain coping efficacy in children; Has shown |
| | good reliability and validity. |

PARENT FACTORS

| | |
|---|---|
| PCS-Parent (PCS-P) | Pain catastrophizing validated in parents of children with chronic pain |
| Adult Responses to | 33-item questionnaire describes negative (Protective, Minimizing) and |
| Children’s Symptoms | positive (Encourage and monitor) parental behaviors towards children |
| (ARCS) | aged 8-18 years, with good internal consistency. |

OUTCOMES and FOLLOW-UP DATA (2-3 months and 4-6 months)

| | |
|---|---|
| Electronic pain diary/ | NRS of average pain intensity based on electronic pain ratings |
| PainDETECT (PD) | for one week, activity and sleep, using free smartphone application |
| | (“Manage my Pain” for Android and “Pain Diary” for iPhone users) and |
| | Twilio PD assesses pain nature, with high sensitivity, specificity and |
| | positive predictive accuracy in pain conditions |
| | for nociceptive (<12) and neuropathic pain (>19). |
| Functional disability | 15-item scale that assesses the extent to which children experience |
| Index (FDI) | difficulties in completing specific tasks (for eg., walking to the |
| | bathroom, eating regular meals, being at school all day). It been used in |
| | children with chronic pain and postsurgical pain. |
| Pediatric Quality of Life | This is a validated measure of QL in children. 23-item questionnaire that |
| Inventory 4.0 Generic | assesses physical, emotional, social, and school functioning. Items are |
| Core Scales (PedsQL) | reverse scored and linearly transformed to a 0-100 scale. Provides |
| | Psychosocial and Physical Health Summary subscores. |

Additionally, psychophysical test data, as described in Table 3, was collected preoperatively. Since the order of the tests may make a difference to results and to prevent bias, the less dynamic short tests—TPD and pressure pain (PPT) order based on ID (odd/even), are randomized and then TS is performed. CPM (a more dynamic test) is performed last since there is a risk of carryover with CPM.

TABLE 3
Psychophysical Tests Performed and Rationale for Performance in Study

| | Procedure and Measurement |
|---|---|
| Pressure pain threshold (PPT) | PPT is assessed with a digital, handheld, clinical grade pressure |
| Differences in pressure pain have | algometer (Algomed, Medoc), over the subject’s trapezius muscle at |
| been reported in a number of pain | the upper back, approximately 5 cm lateral to the C8 spinous process |
| conditions and following surgery due | on non-dominant side at approximately 30 kPa/s, until first sensation |
| to changes in the threshold of | of pain is detected, which the participant indicates by pressing a |
| nociceptors within muscle tissue, and | button, and the device records the pressure. Participants are asked to |
| have been used as an experimental | rate perceived intensity and pleasantness/unpleasantness of each |
| method to assess changes in pain | pressure stimulus using NRS. To ensure patient safety, cut-off |
| sensitivity. | pressure is observed (400 kPa). This process is repeated 3 times, |
| | with 20 seconds between measurements, the average pressure will |
| | be taken as the patient's PPT. |
| Conditioned pain modulation | PPT is assessed before (pre-PPT) and during immersion (post-PPT) |
| (CPM) | of participant’s contralateral hand in a cold-water bath (10.0 ± |
| CPM is a reflection of engagement | 4.0° C.). CPM response is the difference between PPT before and |
| (or lack) of endogenous pain | after cold water hand immersion (positive values reflect pain |
| modulatory mechanisms from | sensitization); At about 20 seconds into immersion, participants |
| descending control pathways from | provide a rating of cold pain intensity and unpleasantness. At about |
| the brainstem. Studies have shown | 30 seconds into the immersion, PPT is re-assessed. The participant is |
| that post-surgical pain alleviation is | able to remove his/her hand from cold water after the pressure |
| accompanied by improvement of | stimuli are assessed on the contralateral site. Final cold pain |
| pro-nociceptivity, with transition | intensity and unpleasantness rating is also be assessed. Each |
| from less efficient to efficient CPM. | participant is allowed to remove his/her hand anytime it becomes |
| | intolerable, and the withdrawal time noted. This procedure is |
| | repeated up to two (2) immersions. |
| Temporal summation (TS) | For this test, a nylon monofilament (e.g., approximately 60-300 g) is |
| TS reflects changes with the dorsal | used to examine TS on the non-dominant forearm. Participants |
| horn in which repetitive application | provide a pain rating (NRS) following a single contact (~1 seconds) |
| of a stimulus will result in an | of the monofilament applied to the skin, after which they provide |
| increase in the perception of pain. | another pain rating following a series of 10 contacts at a rate of one |
| Exaggerated TS response has been | contact per second. The difference between NRS for the single |
| found to predict postoperative pain | versus multiple contacts reflects temporal summation of mechanical |
| due to enhancement of central neural | pain. |
| processes. | |

Somatosensory assessment is also performed preoperatively based on parameters developed by the German Neuropathic Pain Network as described in Lim et al., (2010) Lancet 380(9859): 2224-60 and King et al., (2004) Journal of Pain 5(7):377-84, the disclosures of which are incorporated herein in their entirety. Somatosensory assessments are performed done prior to surgery by the same examiner at each testing site, to minimize observer bias. Participants are comfortably positioned. To familiarize participants with the test, a standardized set of instructions is read, and practice trials demonstrated on the non-testing site of the participants' bodies, preferably without the parent/legal guardians present to eliminate parental influence. Each mechanical procedure is conducted over sets of trials to derive an average mean measure and/or pain (numerical pain rating, NRS). Each device is wiped and cleaned with 70% alcohol after being used on each participant.

All patients received total intravenous anesthesia (propofol and remifentanil) and midazolam in the intraoperative period followed by standardized doses of patient controlled analgesia (morphine or hydromorphone) postoperatively. Pertinent surgical details (duration, number of vertebral levels fused) and anesthetic data (propofol and remifentanil doses) were collected. In the immediate Postoperative, pain scores (every four hours), doses of morphine equivalents administered over on postoperative days (POD) one and two were recorded.

After hospital discharge, at 6-12 months, patients were asked to rate their average pain score (NRS) over the previous week and open-ended questions about the nature and site of pain, use of medications/alternative therapies/physician consults for pain, and functional disability (FDI). Briefly, questions (pain score, anxiety and medication dose/use over 24 hours) are sent at 6 PM every day until no opioid use is reported for 3 consecutive days. These follow up questionnaires are administered through Twilio messaging links via REDCap, electronic diary or phone by a trained research coordinator. Typically, opioid use decreases by about 2-3 weeks. Documentation about opioid use after the texting period was obtained through questionnaires administered at 2-3 months and 4-6 months after surgery. The questionnaires are administered in a standard fashion, without prompting answers, giving subjects time to think. Patients were asked to rate their current pain score (NRS) at rest and with daily activity, maximum pain score over the previous week, nature and site of pain, medications/alternative therapies/physician consults for pain. At least 3 phone call attempts at all provided contact numbers to ensure contact, use of monthly reminders, and incentives to facilitate retention and future follow-up are performed. Rate of prescription refills and electronic medication monitoring are alternative strategies to obtain objective opioid use data when contact with patient post-surgery is lost.

Outcomes evaluated included a a) binary outcome: CPSP which was determined based on a cut-off of pain score>3/10 on an 11-point Numerical rating scale (range 0-10) at 6-12 months after surgery. This cut-off was used as NRS pain scores>3 (moderate/severe pain) at three months was a known predictor for persistence of pain, associated with functional disability.

Maladaptive coping strategies in children and negative parent responses affect chronic pain conditions in children. Three coping sub-scales from Pain Coping Scale (approach, behavioral distraction, emotion-focused avoidance) and 3 parenting factors (Protection, Minimizing, Encouragement) from Adult Response to Child's Symptoms are considered for association with CPSP. PROMIS domains have shown construct validity and responsiveness to change in children (ages 8-18) with chronic pain; as suc, pain Interference and Depression is also assessed.

Descriptive statistics (mean and standard deviation for continuous, and frequency and percentage for categorical variables) were calculated for all study variables. Additionally, univariate association between independent variables and outcomes (primary and secondary) were examined using 2-sample t-tests or Wilcoxon rank-sum test, ANOVA or Kruskal Wallis, Spearman or Pearson correlation coefficient, and chi-square or Fisher's exact tests, as appropriate. For each outcome variable, linear (for continuous outcome) or logistic (for binary CPSP outcome) regression models with one primary independent variable of interest at a time were conducted, adjusting for covariates. To compare QST, data are calculated for each individual QST variable or by z transformation as described in Baron et al., (2017) Pain, 158(2):261-72, the disclosure of which is incorporated herein in its entirety. Z scores of zero represent a value corresponding to the mean of the non-CPSP cohort for that measure; z scores above “0” indicate a gain of function when the patient was more sensitive to the test stimuli compared with controls, z scores below “0” indicate loss of function referring to a lower sensitivity of the patient. For each outcome variable, linear regression models with one primary independent variable of interest at a time are conducted, adjusting for covariates. Increase in coefficient of determination (R²) due the additional primary independent variable of interest were reported. To build a multivariable model for each outcome, factors associated at p<0.10 in univariate analysis are subject to testing of multicollinearity (and removed if collinearity exists) and then entered into multivariable regression models. Stepwise selection was used to derive a final model for each outcome where only variables with a p<0.05 are retained. R² is reported for the final models. A random effect is included in all regression models to account for the cluster effect of study center. As a secondary analysis, pairwise correlation between continuous and categorical outcomes are examined using either Spearman or Pearson correlation coefficient as appropriate. Cronbach's alpha is used to assess internal consistency of the questionnaires. In general, an alpha of >0.7 is acceptable.

Demographics and summary of the variables examined for the prospective cohort are given in Table 4 where the following abbreviations were used: CASI: Childhood anxiety sensitivity index; PCS: Pain catastrophizing scale; AUC: Area under curve of pain scores over postoperative days (POD) 1 and 2; CPSP: Chronic post-surgical pain; FDI: Functional disability index.

TABLE 4
Baseline and pain follow-up characteristics of the surgical cohort

| | | | |
|---|---|---|---|
| | CPSP Yes | CPSP No | P- |
| Variable | (N = 53) | (N = 78) | value |

Demographics

| Sex F % | 75.4% | | 81% | | 74.4% | | 0.365 |
|---|---|---|---|---|---|---|---|
| Race (White %) | 81.8% | | 77.4% | | 84.6% | | 0.292 |
| | | | | | | | P- |
| | Mean | Std Dev | Mean (SD) | | Mean (SD) | | value |
| Weight (Kg) | 57.446 | 15.256 | 56.3 | (14.2) | 57.0 | (14.5) | 0.781 |
| Age (Yrs) | 14.488 | 1.840 | 14.7 | (1.8) | 14.5 | (1.8) | 0.462 |

Preoperative characteristics

| Preoperative pain | 0.596 | 1.282 | 0.3 | (0.5) | 0.1 | (0.3) | 0.037 |
|---|---|---|---|---|---|---|---|
| score | | | | | | | |
| CASI | 28.552 | 5.531 | 30.6 | (5.6) | 26.8 | (4.9) | 0.003 |
| PCS_child | 16.230 | 10.354 | 18.2 | (11.1) | 14.4 | (9.2) | 0.122 |

Surgical/anesthesia characteristics

| Surgical duration | 4.816 | 1.232 | 5.0 | (1.4) | 4.8 | (1.2) | 0.376 |
|---|---|---|---|---|---|---|---|
| No. vertebral levels | 11.506 | 1.969 | 11.0 | (2.3) | 11.6 | (1.9) | 0.115 |
| fused | | | | | | | |
| Propofol dose | 71.791 | 27.186 | 79.5 | (27.0) | 73.7 | (28.7) | 0.238 |
| mg/kg | | | | | | | |
| Remifentanil dose | 113.911 | 40.891 | 118.6 | (41.5) | 115.2 | (44.2) | 0.563 |

mcg/kg

Acute postoperative pain characteristics

| | | | | | | | |
|---|---|---|---|---|---|---|---|
| AUC POD 1-2 | 200.327 | 73.490 | 222.7 | (75.9) | 196.7 | (66.8) | 0.053 |
| Morphine Meq | 1.626 | 0.747 | 1.6 | (0.7) | 0.8 | (0.1) | 0.065 |

POD 1-2 mg/kg

Pain follow-up at 6-12 months

| CPSP Y/No % | 53/78 (40.5%) | | | | | | |
|---|---|---|---|---|---|---|---|
| FDI score | 4.485 | 5.321 | 6.7 | (5.9) | 2.3 | (4.0) | 0.002 |
| Pain score (NRS) | 2.240 | 2.457 | 4.6 | (2.0) | 0.6 | (1.0) | <0.001 |

CPSP outcome was determined for 131 of the 171 patients (loss to follow up of about 23%). The characteristics of the cohort that was lost to follow up and those followed for 6-12 months were examined for all pertinent measures included in the models and did not find any significant differences in terms of age (p=0.390), sex (p=0.361), race (0.906), CASI (p=0.364), surgical duration (p=0.322) and preoperative pain (p=0.879). Incidence of CPSP was found to be 53/131 (40.4%).

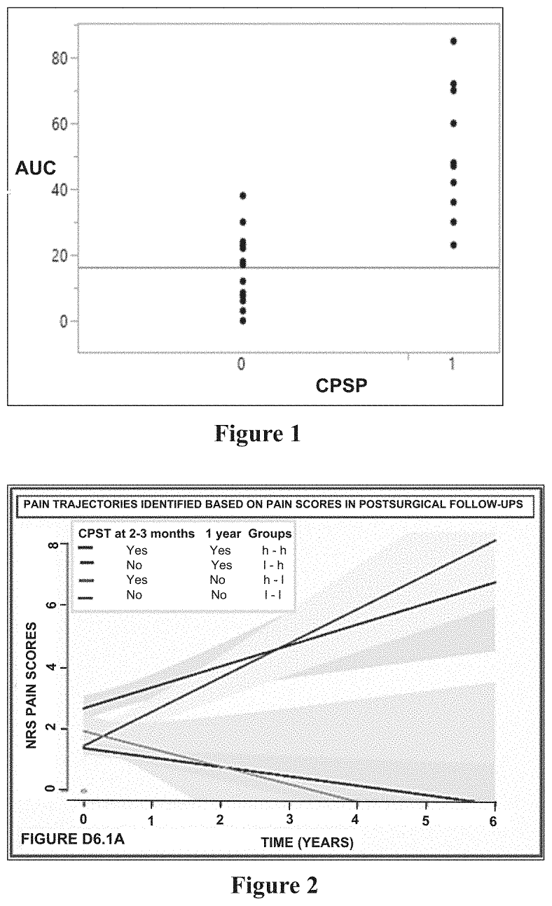

Although 83% of the cohort had no preoperative pain, there was a 37.8% incidence of post-surgical pain at 2-3 months, and 41.8% at one year after spine fusion. FDI scores were higher (p=0.001) and PedsQL scores were lower (P=0.001) in patients with CPSP vs. those without. CPSP was defined as numerical rating scale (NRS) pain scores >3/10 at 6-12 months. AUC of repeat pain scores over 6 and 12 months measured cumulative pain experience over time after surgery and was well correlated with CPSP (p<0.0001). FIG. 1.

Pain trajectories measured for over six years after spine surgery were evaluated in the spine cohort. Of the 66% of patients who developed CPSP, they reported high pain scores throughout the 6 years follow-up (h-h) and those who did not had low scores throughout (1-1). However, 33% of those who developed CPSP had unexpected trajectories. FIG. 2.

Higher anxiety sensitivity causes fear of pain and avoidance behavior leading to chronic pain and disability. Accordingly, PPST, (composite assessment of pain catastrophizing, fear of pain, anxiety, and depressive symptoms) was evaluated in the cohort. On univariate analysis, PPST was positively associated with CPSP (p<0.002; β: 0.95, SE 0.26) functional disability (p<0.0001; β2.92, SE 0.43), and negatively associated with QOL scores (p<0.0001; β-20.72, SE 3.64).

Child anxiety and pain catastrophizing, parent anxiety and catastrophizing were evaluated as predictors of CPSP. In multivariate regression models, CASI was identified as significant psychosocial predictor of CPSP. Table 5.

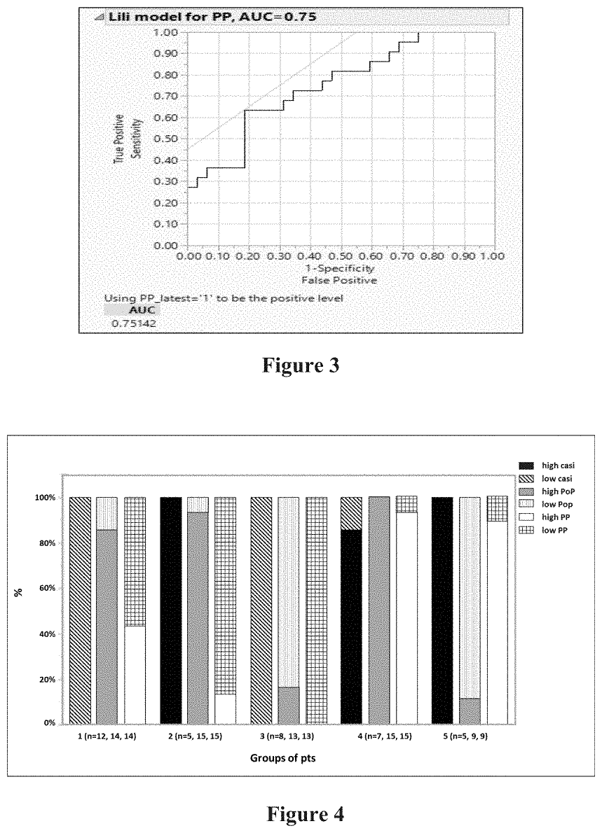

To assess further assess the diagnostic ability of psychological and perioperative factors as predictors phenotypes of risk for CPSP, a multiple regression model with psychosocial factors was developed which predicted CPSP with 75% predictive accuracy as determined by receiver operating characteristic (ROC) curves. FIG. 3.

TABLE 5
Psychosocial Predictors of CPSP

| Parameter | OR | 95% CI | p-value |
|---|---|---|---|
| Surgical duration | 2.16 | 1.17-4.00 | 0.014 |
| Pain scores postoperative | 1.02 | 1.01-1.03 | 0.003 |
| days 1 & 2 | | | |
| CASI | 1.24 | 1.09-1.42 | 0.002 |

Next, data from the psychosocial tests were subjected to Hierarchical cluster analysis (HCA) to group patients with similar structure into clusters of phenotypes. Briefly. HCA was used to group patients with similar structure into phenotypes based on 3 factors: 1) pain character (AUC and nature—inflammatory or neuropathic based on PainDETECT score (1-12: nociceptive pain, and 19-38: neuropathic pain is likely)); 2) psychophysical characteristics; and 3) opioid use (dose and days of use over weeks after surgery). For cluster characteristics, continuous values of the measures or dichotomize by high tertile and low tertile for discordant phenotypes was used. Before clustering, a Principle Component Analysis (PCA) was performed to reduce dimensionality of data. A screen plot was analyzed to detect an “elbow” that suggested the number of PCs for entering hierarchical clustering. A wide variety of hierarchical clustering techniques were applied to achieve robust and meaningful data segregation, including divisive and agglomerative algorithm, Ward's and other methods for the linkage criterion, Euclidean, Manhattan and correlation-bases distances for similarities. To detect an optimal number of clusters, heatmaps and dendrograms were visually inspected and R software (package NbClust) that identifies optimal number of clusters using multiple indexes was employed. These clusters were evaluated in the context of prior knowledge to identify the most parsimonious clustering. HCA and PCA were performed in R software (package factoextra v1.0.3). Cophenetic correlation, which was the Pearson correlation between actual and predicted distances based on clustering approach as calculated. A value of 0.75 or above was needed for goodness of cluster fit.

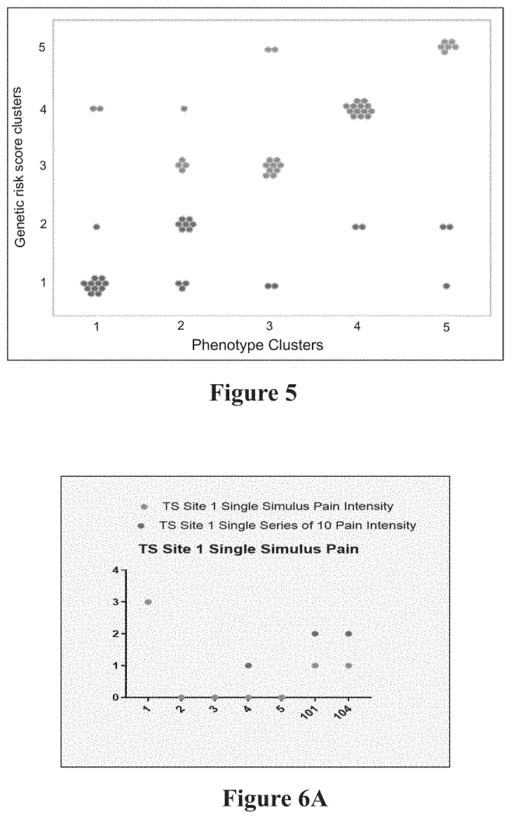

Hierarchical cluster analysis identified five phenotype dusters based on high and low risk for acute postoperative pain, CPSP and CASI. FIG. 4. PRS were able to differentiate the phenotype clusters on co-clustering, thus indicating that unique genotypes determine phenotype sub-groups. FIG. 5.

### Example 3. Assessment of Psychosocial and Perioperative Factors as Predictors of CPSP in a Pectus Cohort

A cohort of 7 children undergoing Pectus surgery under standard anesthesia/pain protocols was selected according to the protocol in Example 1. Preoperative data on demographics (sex, age, race), weight, home medications, and Haller's Index (ratio of the measure of the transverse diameter of the chest, divided by the sagittal measure of the distance from the sternum to the vertebral body, reported on routine preoperative CT/MRI chest—in the pectus cohort), were collected from the cohort. NRS and the psychosocial variables PCS, FDI, CASI were assessed in the pectus cohort in the same manner as described in Example 2 for the scoliosis cohort.

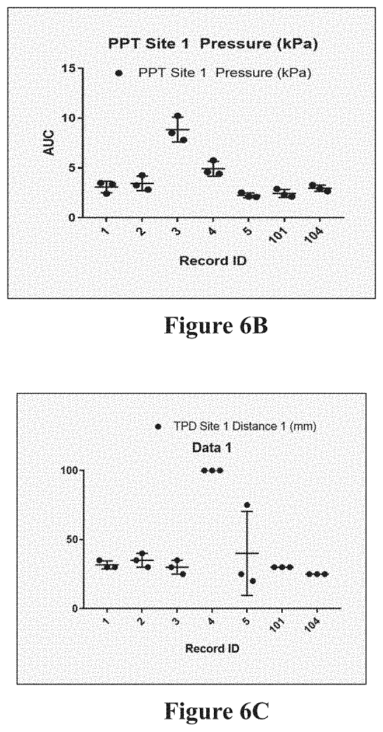

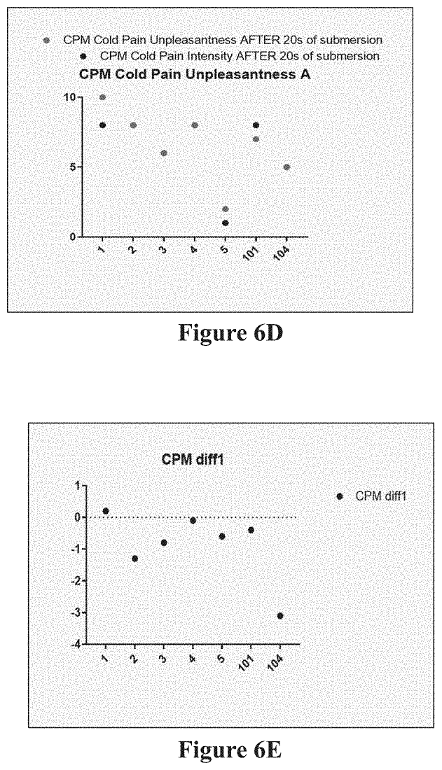

Similarly, psychophysical sensory test data was collected in same manner as described in Example 2 for the scoliosis cohort in 7 patients before pectus surgery. Briefly, two-point discrimination (TPD), pressure pain threshold testing (PPT), conditioned pain modulation (CPM), temporal summation (TS), and pain scores were collected and mapped over 2-6 months postoperatively. Pain intensity after hand insertion at 20 seconds and withdrawal time noted. FIGS. 6A-6E shows the preoperative sensory testing differences observed in each patient.

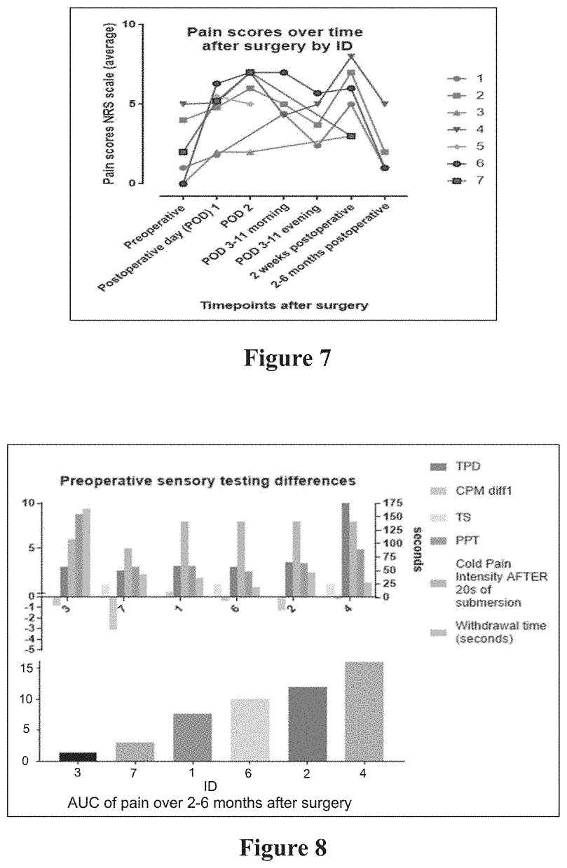

Pain scores were collected and mapped over 2-6 months postoperatively through REDCap data collection using Twilio messaging and phone calls/emails. AUC under pain trajectories were calculated (except for patient ID NO. 5 who withdrew from study). FIG. 7. Data from the preoperative sensory tests for each patient were subjected to quantitative sensory testing (QST) to refine an endophenotype characterization. The resulting sensory testing profiles of each patient along with AUC are presented in FIG. 8. Sensory profiles of patient ID NO. 3 and patient ID NO. 4, (lowest and highest AUC respectively) were almost mirror images. Direction of the responses was as expected. A closer look at the association of CPM response with AUC showed impaired CPM response (less negative pain response after cold modulation) was associated with higher AUC (p=0.02) indicating decreased descending inhibition of nociception, predicting a high pain phenotype (enhanced TS).

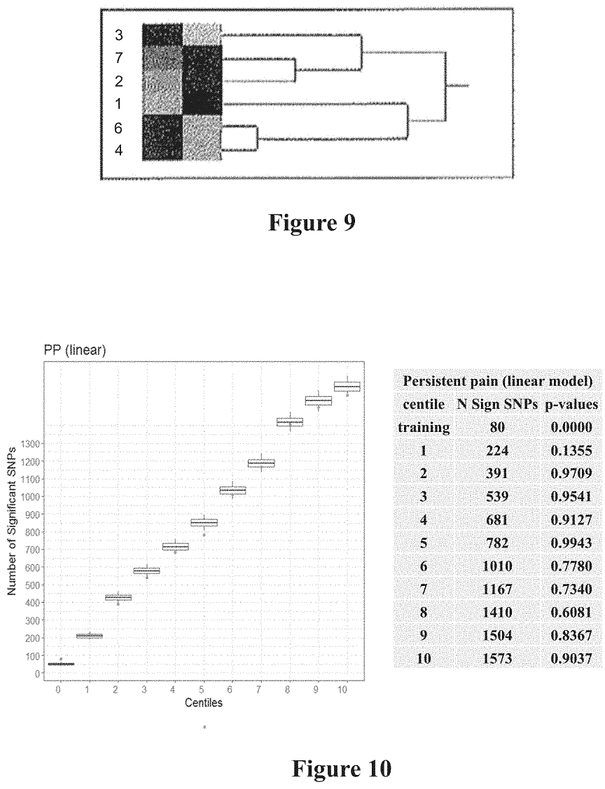

Clusters of phenotypes were identified using RCA (in the manner described in Example 2) after decreasing dimensionality via principal component analysis. FIG. 9. Cluster 1 (patient ID NOs. 3, 7, 2) and cluster 2 (patient ID NOs. 1, 4, 6) differed mainly in CPM responses. These data demonstrate feasibility of preoperative sensory testing in this population and risk clustering predicted by sensory testing risk profiles.

### Example 4. Identification of a Polygenic Risk Score (PRS) Predictive for CPSP Risk

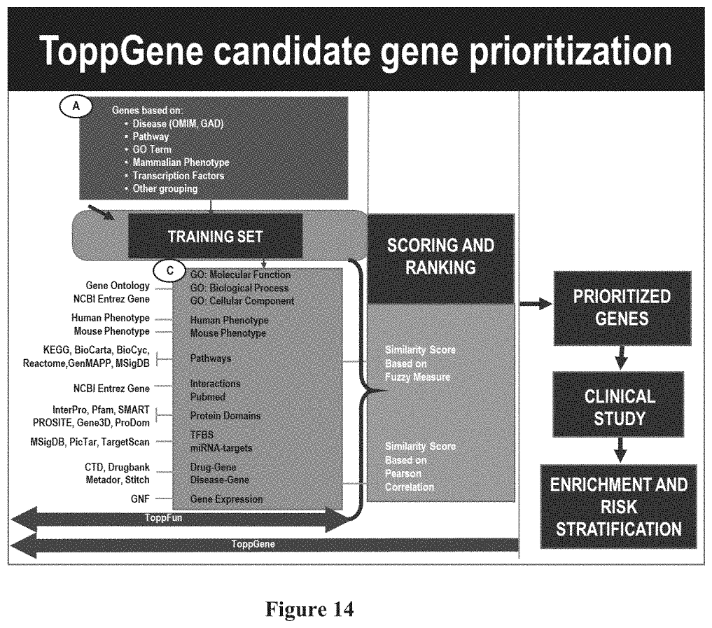

Genetic factors influence individual differences in pain perception but the effect these factors have on pain perception is small and causal variants are difficult to detect. It is possible that it is not just one genetic factor contributing to the risk of CPSP, but instead the combined effects of a large number of susceptibility loci may become large enough to be useful for targeted risk prediction and prevention. A polygenic risk score (PRS) can be used to model these weak contributions, where an individual's genetic risk is the sum of all their risk alleles weighted by significance of the corresponding allele in genome wide association studies. To identify polygenic risk scores for CPSP, an approach outlined in FIG. 14 was followed.

(i) Identification of “Training” and “Candidate” Genes for Psychosocial and/or Perioperative Phenotypes.

To generate the “training set” of genes for each psychosocial and/or perioperative phenotype observed in either the scoliosis cohort or pectus cohort, a comprehensive literature search limited to human studies using electronic databases including PubMed and MEDLINE (according to the Preferred Reporting Items of Systematic Reviews and Meta-Analyses (PRISMA) guidelines), was conducted from January 2001 to December 2017, using the following search terms: (“postoperative pain” OR “postsurgical pain” OR “post-operative pain” OR “post-surgical pain” OR “postoperative analgesia” OR “postoperative opioid” OR “CPSP”) AND (genetic association OR polymorphism OR variant OR “genotype” OR “Genome wide association” OR “SNP”). Searches were limited to English language articles and human studies (including clinical study, clinical trial, multicenter study, observational study and twin study) using filters. The search was conducted from 2001/01/01 to 2017/12/31. Detailed results of this review including description of studies, genes, variants and outcomes studied are detailed in Chidambaran et al., (2019) J Pain, May 23. pii: S1526-5900(19)30079-3, the disclosure of which is incorporated herein in its entirety. Ultimately, the literature search identified 31 “training” genes associated with postoperative pain and CPSP in humans. Those “training” gene were: COMT (rs6269, rs4633); GCH1 (rs3783641, rs8007267); COMT rs4680; ABCB1 C3435T; 5HTR2A rs6311; IFNG1 (rs2069727, rs2069718); IL1R1 rs3917332; IL1R2 rs11674595; IL4 rs2243248; IL10 (rs3024498, rs1878672, rs3024491); IL13 (rs1881457, rs1800925, rs1295686, rs20541); NFKB1 rs4648141; HLA-DRB1*4 and DQB1/03:02; PRKCA rs887797; CDH18 rs4866176; TG rs1133076; ATXN1 rs179997; DRD2 (rs4648317, rs12364283); NFKB1A rs8904; GCH1 rs4411417; CHRNA6 rs7828365; KCND2 (rs17376373, rs702414, rs802340, rs12706292); KCNJ3 (rs6435329, rs11895478, rs3106653, rs3111006, rs12471193, rs7574878, rs12995382); KCNJ6 rs2835925; KCNK3 (rs1662988, rs7584568); KCNK9 rs2014712; CACNG2 (rs4820242, rs2284015, rs2284017, rs2284018, rs1883988); COMT (rs4680, rs6269); P2X7R (rs208294, rs208296, rs7958311); KCNS1 (rs734784, rs13043825); TNF alpha rs1800629; and, GCH1 rs8007627.

Next, the ToppFun application of the Transcriptome Ontology Pathway PubMed based prioritization of Genes (ToppGene) Suite for candidate gene prioritization was used similar to that described in Chen et al., (2009) Nucleic Acids Research 37 (Web Server issue):W305-311, the disclosure of which is incorporated herein in its entirety. 1310 “candidate” genes enriched for CPSP were identified and prioritized using Toppgene suite based on functional enrichment using several gene ontology annotations, and curated gene sets associated with mouse phenotype-knockout studies.

Training and candidate gene sets together formed the case set of genes whose variants were used for association analyses as described herein.

(ii) DNA Collection and Genotyping.

Blood was drawn upon intravenous line placement for genotyping from 171 adolescents undergoing spine fusion (the Scoliosis Cohort described in Examples 1 and 2). DNA degradation or modification caused by environmental factors and by pathogens present in the sample will be prevented by using DNA/RNA Shield which preserves the genetic integrity of samples at ambient temperatures (cold-free) for >2 years.

Deoxy ribonucleic acid (DNA) was isolated and purified using standard procedures on the same day, frozen at −20° C. Concentration and purity of genomic DNA was determined using a Thermo Scientific NanoDrop spectrophotometer, to insure a minimum of 400 ng of genomic DNA free of contaminants was obtained to be used in genotype assays. Genotyping was done using the Illumina Human Omni5 v41-0 array (85 patients), Human Omni5Exome v41-1 (33 patients) and Infinium Omni5-4-v1 (53 patients). Arrays were changed due to availability of new array which had more SNPs and functional ones.

(iii) Genetic Association Analyses.

Analyses were conducted using SAS 9.4 (SAS, Cary, N.C.) and R. Prior to genetic analyses, 121,301 SNPs from the sex chromosome, chromosome zero, mitochondrial, indels and other were excluded from analysis. Quality of SNP calls from the chip were also evaluated. SNPs were assessed for Hardy-Weinberg equilibrium (HWE) by means of goodness of fit χ2 test. SNPs deviating HWE (p<0.0001), whose MAF is less than 90%, or whose call rates fall below 95% were excluded. Thresholds for quality control for call rates at individual and SNP levels were 99% and 90%, respectively. Low-frequency variants were also excluded, threshold for minor allele frequencies was 10%. SNPs in high linkage disequilibrium (LD) (80%) were pruned out in PLINK using the command --indep-pairwise 50 5 0.8. SNPs from autosomal chromosomes only were selected for analysis and were annotated using ANNOVAR software in a manner similar to that described in Wang et al., (2010) Nucleic Acids Research 38(16):e164-e164, the disclosure of which is incorporated herein in its entirety. SNPs located in intergenic regions and not associated with a specific gene according to ANNOVAR annotation were also excluded prior to analysis. Prior to genetic analyses, cryptic relatedness was checked using Graphical Representation of Relationship (GRR), in a similar manner as described in Abecasis et al., (2001) Bioinformatics Applications Note 2001; 17(8):742-743, the disclosure of which is incorporated herein in its entirety. Principal component analysis was employed to confirm European and African continental ancestry using 482 validated ancestry informative markers. Concordance with self-reported race was >95%.

To identify significant SNPs, we used logistic and linear models for association of each SNP with CPSP and the pain score at 6-12 months as outcomes. In all association tests we used an additive genetic model in which major homozygotes were coded as 0, heterozygotes as 1, and minor homozygotes as 2. Surgical duration, CASI and acute postoperative pain over 48 hours (area under curve of pain scores over time) were used as covariates, based on our previous multiple regression model which did not include genetics. PLINK v.07 was used for association tests.

There were 4,186,587 variants on the exome chip initially and 542,313 variants remained after exclusion. After quality control, of the 542,313 variants that were analyzable, 61,348 variants were annotated to training/candidate genes of interest. Pruning was then done based on Linkage disequilibrium (r²=0.5) to remove variants that were highly linked and minimize correlation issues. Thus, a set of 33,104 variants were finally included for association analyses in our cohort.

(iv) Gene Enrichment Analyses:

Case gene variants for each outcome (training and prioritized candidate genes) were analyzed as sequence of cumulative sums of ranked variant sets with 10% increment. The first addend in each sequence was the training gene variant set. For each cumulative sum we compared the number of associations in our case sets that met the p<0.05 threshold to the number of associations meeting the same criteria in over 10,000 matched runs of our control set of genes. SNPs from the control set were selected in the same ratio for MAF as it was observed in the case set. Specifically, we used MAF bands as follows: 10-15%:15-20%:20-30%:30-50%. Empirical p-values of resampling tests were computed as follows: we calculated how many samples out of 10,000 had the number of significant SNPs equal to or greater than the number of significant SNPs from the case set and divided this number by 10,000. Training and ranked genes that formed the earliest cumulative group where a number of significant SNPs were greater than in the matched control group were considered as a minimal set of variants enriched for associations with corresponding outcomes.

As shown in FIG. 10, the box plots represent the number of significant single nucleotide polymorphisms (SNPs) in the 10,000 runs of control gene SNPs. The upper and lower bounds of the box represents the 75^{th} and 25^{th} percentile, respectively, and error bars represent the 5^{th} and 95^{th} percentiles. Vertical axis represents the number of SNPs and the horizontal axis is the centiles of the ranked case genes as described in methods using ToppGene. The Red dot represents the number of positive SNPs in the case set of genes. When the red dot was above the 95^{th} centile of the box plot whisker on the figure, this indicated that more case genes were associated with behavioral outcomes than expected, indicating the case genes were enriched compared to control-gene SNPs. At the 10^{th} centile and above, there were more case-gene SNPs significantly associated with behavioral outcomes than in the 10,000 runs of the control-gene SNPs, indicating gene enrichment. Because FIG. 10 demonstrates main effects controlling for the group, these findings indicated that a complex network of genes/polymorphisms was associated with increased risk of CSPS.

Case genes for each outcome were analyzed as sequence of cumulative sums of ranked genes with 10% increment. The first addend in each sequence was a number of training genes. For each cumulative sum we compared the number of associations in our case sets that met the p<0.05 threshold to the number of associations meeting the same criteria in over 10,000 matched runs of our control set of genes. After adjusting for covariates, compared to control sets, there was enrichment of SNP associations in training set for CPSP. Training and ranked genes that formed the earliest cumulative group where a number of significant SNPs were greater than in the matched control group were considered as a minimal set of variants enriched for associations with corresponding outcomes. The number of genes and SNPs that ere included in these significant case sets were 12 genes (80 SNPs) for CPSP ((ATXN1 (29); CACNG2 (2); CTSG (2); DRD2 (1); HLA-DQB1 (3); IL10 (1); KCNA1 (1); KC ND2 (5); KCNJ3 (3); KCNJ6 (9); KCNK3 (2); PRKCA (22)). The complete list of the 80 significant SNPs is provided in Table 6.

TABLE 6
Cumulative Group of Significant SNPs

| SNP kgp | Reference SNP | | Functional |
|---|---|---|---|
| Identifier | ID number | Gene | Consequence |
| rs4791072 | rs4791072 | PRKCA | intronic |
| kgp7438896 | rs11079653 | PRKCA | intronic |
| kgp7619382 | rs1543359 | ATXN1 | intronic |
| rs1570487 | rs1570487 | ATXN1 | intronic |
| kgp11151933 | rs17376373 | KCND2 | intronic |
| kgp8949113 | rs61131185 | ATXN1 | intronic |
| kgp3695992 | rs12198202 | ATXN1 | intronic |
| kgp10086344 | rs62069959 | PRKCA | intronic |
| kgp10839938 | rs3024500 | IL10 | downstream |
| kgp8772329 | rs744214 | PRKCA | intronic |
| rs607138 | rs607138 | ATXN1 | intronic |
| kgp11011645 | rs1018388 | ATXN1 | intronic |
| rs493352 | rs493352 | ATXN1 | intronic |
| rs10488301 | rs10488301 | KCND2 | intronic |
| rs7225452 | rs7225452 | PRKCA | intronic |
| kgp14182269 | rs202146909 | KCNJ3 | intronic |
| kgp8125121 | rs6504424 | PRKCA | intronic |
| kgp2649254 | rs2237208 | ATXN1 | intronic |
| kgp10653910 | rs9754467 | CACNG2 | intronic |
| rs3812196 | rs3812196 | ATXN1 | intronic |
| rs7125415 | rs7125415 | DRD2 | intronic |
| kgp11224094 | rs973753 | PRKCA | intronic |
| kgp3415849 | rs562760 | ATXN1 | intronic |
| rs932411 | rs932411 | ATXN1 | intronic |
| kgp2759470 | rs61687889 | PRKCA | intronic |
| kgp920275 | rs56399646 | PRKCA | intronic |
| rs9635753 | rs9635753 | PRKCA | intronic |
| kgp10620928 | rs9914792 | PRKCA | intronic |
| rs1662987 | rs1 662987 | KCNK3 | intronic |
| kgp11700085 | rs7220480 | PRKCA | intronic |
| rs2927 | rs2927 | ATXN1 | intronic |
| rs7281804 | rs7281804 | KCNJ6 | intronic |
| rs9914723 | rs9914723 | PRKCA | intronic |
| rs12665284 | rs12665284 | ATXN1 | intronic |
| rs2836019 | rs2836019 | KCNJ6 | intronic |
| kgp4018125 | rs4716080 | ATXN1 | intronic |
| rs2891519 | rs2891519 | KCNK3 | downstream |
| rs1150635 | rs1150635 | ATXN1 | intronic |
| rs3812204 | rs3812204 | ATXN1 | intronic |
| kgp2569546 | rs1957523 | CTSG | intronic |
| rs9903921 | rs9903921 | PRKCA | intronic |
| kgp10084577 | rs77929576 | KCNJ3 | intronic |
| kgp4011063 | rs713952 | CACNG2 | intronic |
| kgp7502730 | rs4716060 | ATXN1 | intronic |
| kgp4976858 | rs179939 | ATXN1 | intronic |
| kgp10121398 | rs1992701 | KCNJ3 | intronic |
| kgp7164508 | rs35105653 | PRKCA | intronic |
| kgp9743316 | rs35210572 | ATXN1 | intronic |
| rs531089 | rs531089 | ATXN1 | intronic |
| kgp14078813 | rs200369418 | PRKCA | intronic |
| kgp679476 | rs2850125 | KCNJ6 | intronic |
| kgp5970168 | rs227912 | PRKCA | intronic |
| kgp8464416 | rs58162388 | PRKCA | intronic |
| rs9890911 | rs9890911 | PRKCA | intronic |
| kgp4691641 | rs11079656 | PRKCA | intronic |
| rs1892681 | rs1892681 | KCNJ6 | intronic |
| rs11088404 | rs11088404 | KCNJ6 | intronic |
| kgp8911945 | rs202032703 | ATXN1 | intronic |
| rs10949375 | rs109493 75 | ATXN1 | intronic |
| kgp4964195 | rs6459476 | ATXN1 | intronic |
| kgp9114806 | rs9273371 | HLA-DQB1 | downstream |
| kgp10396210 | rs858018 | KCNJ6 | intronic |
| rs13048511 | rs13048511 | KCNJ6 | intronic |
| rs3828867 | rs3828867 | ATXN1 | intronic |
| kgp10716887 | rs7809109 | KCND2 | intronic |
| kgp9238504 | rs7280905 | KCNJ6 | intronic |
| kgp7930082 | rs12945884 | PRKCA | intronic |
| rs631661 | rs631661 | ATXN1 | intronic |
| rs2237198 | rs2237198 | ATXN1 | intronic |
| kgp7596948 | rs67881942 | KCND2 | intronic |
| rs11754994 | rs11754994 | ATXN1 | intronic |
| rs12878578 | rs12878578 | CTSG | upstream |
| rs2032090 | rs2032090 | KCNJ6 | intronic |
| kgp9142401 | rs1001211 | ATXN1 | intronic |
| kgp6614124 | rs17843723 | HLA-DQB1 | intronic |
| kgp6691641 | rs10774289 | KCNA1 | upstream |
| kgp9757346 | rs17142908 | KCND2 | intronic |
| kgp4959086 | rs16878842 | ATXN1 | intronic |
| kgp10569724 | rs1049055 | HLA-DQB1 | UTR5 |
| kgp2636165 | rs7218425 | PRKCA | intronic |

To minimize risk of overfitting and identify the minimal number of SNPs for the PRS, we used penalized regression with least absolute shrinkage and selection operator (LASSO) in R software (package glmnet) in a manner similar to that described in Friedman et al., (2010) J Stat Softw 33(1):1-22, the disclosure of which is incorporated herein. A controlling penalty parameter lambda was selected via cross-validation approach. After least absolute shrinkage and selection operator (LASSO), the final set included 9 genes and 24 variants. Chromosomal location, genetic annotation, function, minor allele frequency, Odds ratios for CPSP and beta for NRS at 6-12 months with p-values for the LASSO selected variants are provided in Table 7 (where PRKCA (protein kinase C alpha); DRD2 (dopamine receptor D2); ATXN1 (ataxin 1); KCNJ3 (potassium voltage-gated channel subfamily J member 3); CACNG2 (calcium voltage-gated channel auxiliary subunit gamma 2); CTSG (cathepsin G); KCNJ6 (potassium voltage-gated channel subfamily J member 6); KCNK3 (potassium two pore domain channel subfamily K member 3); KCND2 (potassium voltage-gated channel subfamily D member 2); #Linear regression coefficients were used to calculate weighted polygenic risk scores; *Risk allele).

TABLE 7
Genetic variants and risk alleles with regression coefficients included in the determination
of polygenic risk score for prediction of chronic post-surgical pain.

| | Observed | Observed | | #Linear | p-value | OR | OR | P value |
|---|---|---|---|---|---|---|---|---|
| | major | Minor | | regression | linear | 95% | 95% | logistic |
| SNP | allele | allele | Gene | weight | regression | LCI | UCI | regression |
| rs62069959 | G* | A | PRKCA | 2.299 | 0.001 | 2.47 | 40.2 | 0.001 |
| rs7125415 | G | A* | DRD2 | 1.657 | 0.034 | 1.55 | 17.73 | 0.008 |
| rs61131185 | A | G* | ATXN1 | 1.524 | 0.011 | 1.66 | 12.7 | 0.003 |
| rs12665284 | G* | A | ATXN1 | 1.481 | 0.041 | 1.03 | 18.85 | 0.046 |
| rs202146909 | A* | G | KCNJ3 | 1.414 | 0.042 | 1.25 | 13.59 | 0.02 |
| rs493352 | G* | A | ATXN1 | 1.242 | 0.031 | 1.39 | 8.62 | 0.008 |
| rs9754467 | A* | G | CACNG2 | 1.166 | 0.032 | 1.17 | 8.78 | 0.023 |
| rs1957523 | G | A* | CTSG | 1.103 | 0.023 | 1.24 | 7.3 | 0.015 |
| rs12198202 | A* | G | ATXN1 | 1.064 | 0.005 | 1.35 | 6.2 | 0.006 |
| rs11079653 | T* | A | PRKCA | 0.98 | 0.011 | 0.98 | 7.24 | 0.054 |
| rs1150635 | G | A* | ATXN1 | 0.937 | 0.009 | 1.12 | 5.81 | 0.025 |
| rs2850125 | G* | A | KCNJ6 | 0.936 | 0.046 | 1.12 | 5.78 | 0.025 |
| rs9914723 | G | A* | PRKCA | 0.917 | 0.004 | 0.93 | 6.75 | 0.07 |
| rs7220480 | A | G* | PRKCA | 0.857 | 0.048 | 1.12 | 4.94 | 0.023 |
| rs2891519 | G | A* | KCNK3 | 0.835 | 0.008 | 0.93 | 5.69 | 0.07 |
| rs7809109 | A* | G | KCND2 | 0.833 | 0.045 | 0.75 | 7.09 | 0.147 |
| rs200369418 | A* | C | PRKCA | 0.816 | 0.028 | 1.12 | 4.55 | 0.022 |
| rs3812204 | G | A* | ATXN1 | 0.789 | 0.038 | 0.99 | 4.89 | 0.053 |
| rs4716060 | C | A* | ATXN1 | 0.772 | 0.038 | 1.03 | 4.55 | 0.042 |
| rs531089 | A* | G | ATXN1 | 0.739 | 0.02 | 1.03 | 4.25 | 0.041 |
| rs6459476 | A | C* | ATXN1 | 0.736 | 0.048 | 0.98 | 4.47 | 0.058 |
| rs227912 | A* | G | PRKCA | 0.678 | 0.049 | 0.79 | 4.91 | 0.146 |
| rs744214 | G* | A | PRKCA | 0.634 | 0.017 | 0.81 | 4.37 | 0.139 |
| rs1992701 | G | A* | KCNJ3 | 0.584 | 0.047 | 0.79 | 4.06 | 0.161 |

| | | | | | | Minor |
|---|---|---|---|---|---|---|
| | Reference | Alternative | | | Location | allele |
| SNP | allele | Allele | Function | Chr | (GRCh37) | frequency |
| rs62069959 | C | T | intronic | 17 | 64318923 | 0.196 |
| rs7125415 | C | T | intronic | 11 | 113000000 | 0.126 |
| rs61131185 | A | G | intronic | 6 | 16623387 | 0.322 |
| rs12665284 | G | A | intronic | 6 | 16626066 | 0.146 |
| rs202146909 | T | C | intronic | 2 | 156000000 | 0.193 |
| rs493352 | T | C | intronic | 6 | 16744169 | 0.488 |
| rs9754467 | G | A | intronic | 22 | 37019059 | 0.222 |
| rs1957523 | C | T | intronic | 14 | 25044712 | 0.429 |
| rs12198202 | T | C | intronic | 6 | 16679771 | 0.424 |
| rs11079653 | A | T | intronic | 17 | 64352329 | 0.202 |
| rs1150635 | G | A | intronic | 6 | 16729739 | 0.322 |
| rs2850125 | C | T | intronic | 21 | 39130114 | 0.456 |
| rs9914723 | G | A | intronic | 17 | 64716397 | 0.196 |
| rs7220480 | A | G | intronic | 17 | 64686679 | 0.406 |
| rs2891519 | G | A | downstream | 2 | 26954991 | 0.220 |
| rs7809109 | A | G | intronic | 7 | 120000000 | 0.134 |
| rs200369418 | C | A | intronic | 17 | 64762496 | 0.500 |
| rs3812204 | G | A | intronic | 6 | 16698022 | 0.345 |
| rs4716060 | C | A | intronic | 6 | 16310456 | 0.345 |
| rs531089 | T | C | intronic | 6 | 16706041 | 0.497 |
| rs6459476 | A | C | intronic | 6 | 16618187 | 0.348 |
| rs227912 | G | A | intronic | 17 | 64610729 | 0.246 |
| rs744214 | G | A | intronic | 17 | 64334856 | 0.316 |
| rs1992701 | C | T | intronic | 2 | 156000000 | 0.453 |

(v) Enriched Genomic Pathways:

Toppfun application of Toppgene suite was used to identify top pathways enriched by the genes with significant associations with each phenotype. The pathways enriched by the 9 genes associated with CPSP, based on Bonferroni correction for multiple adjustment cut-offs (p<0.05) are presented in Table 8.

TABLE 8
Genetic pathways enriched by the genes whose variants contributed to
weighted polygenic risk.

| | | -Log | |
|---|---|---|---|
| Pathway | Enriching genes | (p-value) | p-value |
| Heterotrimeric G-protein | DRD2 KCNJ3 KCNJ6 PRKCA | 10 | 1E−10 |
| signaling pathway-Gq alpha and | | | |
| Go alpha mediated pathway | | | |
| Serotonergic synapse | KCND2 KCNJ3 KCNJ6 PRKCA | 10 | 1E−10 |
| Potassium Channels | KCND2 KCNJ3 KCNJ6 KCNK3 | 10 | 1E−10 |
| Neuronal System | CACNG2 KCND2 KCNJ3 KCNJ6 | 10 | 1E−10 |
| | KCNK3 PRKCA | | |
| Dopaminergic synapse | DRD2 KCNJ3 KCNJ6 PRKCA | 10 | 1E−10 |
| Neurotransmitter Receptor | CACNG2 KCNJ3 KCNJ6 PRKCA | 5.9 | 1.26E−06 |
| Binding and Downstream | | | |
| Transmission In The | | | |
| Postsynaptic Cell | | | |
| Oxytocin signaling pathway | CACNG2 KCNJ3 KCNJ6 PRKCA | 5.83 | 1.48E−06 |
| Transmission across Chemical | CACNG2 KCNJ3 KCNJ6 PRKCA | 5.22 | 6.06E−06 |
| Synapses | | | |
| Excitatory synaptic transmission | DRD2 KCND2 | 4.79 | 1.62E−05 |
| Morphine addiction | KCNJ3 KCNJ6 PRKCA | 4.69 | 2.06E−05 |
| Circadian entrainment | KCNJ3 KCNJ6 PRKCA | 4.62 | 2.42E−05 |
| Retrograde endocannabinoid | KCNJ3 KCNJ6 PRKCA | 4.55 | 2.82E−05 |
| signaling | | | |
| Cholinergic synapse | KCNJ3 KCNJ6 PRKCA | 4.42 | 3.84E−05 |
| Cardiac conduction | CACNG2 KCND2 KCNK3 | 4.11 | 7.8E−05 |
| Heterotrimeric G-protein | DRD2 KCNJ3 KCNJ6 | 4.04 | 9.18E−05 |
| signaling pathway-Gi alpha and | | | |
| Gs alpha mediated pathway | | | |
| G protein gated Potassium | KCNJ3 KCNJ6 | 3.97 | 0.000108 |
| channels | | | |
| Inhibition of voltage gated Ca2+ | KCNJ3 KCNJ6 | 3.97 | 0.000108 |
| channels via Gbeta/gamma | | | |
| subunits | | | |
| Activation of G protein gated | KCNJ3 KCNJ6 | 3.97 | 0.000108 |
| Potassium channels | | | |
| Nicotine pharmacodynamics | DRD2 KCNK3 | 3.84 | 0.000145 |
| pathway | | | |
| Inwardly rectifying K+ channels | KCNJ3 KCNJ6 | 3.78 | 0.000166 |

(vi) Polygenic Risk Scores and Multiple Regression Model:

Weighted genetic risk were calculated from the SNPs selected by LASSO. Briefly, SNPs with non-zero coefficients in the LASSO model were selected for PRS calculation. Variants included in the PRS are presented in Table 9.

TABLE 9
SNPs included in PRS calculation.

| Genes | SNPs with p < 0.05 |
|---|---|
| CACNG2 | rs9754467, rs713952 |
| DRD2 | rs7125415 |
| KCND2 | rs17376373, rs10488301, rs7809109, rs67881942, rs17142908 |
| KCNJ3 | rs202146909, rs77929576, rs1992701 |
| KCNJ6 | rs7281804, rs2836019, rs2850125, rs1892681, rs11088404, rs858018, |
| | rs13048511, rs7280905, rs2032090 |
| PRKCA | rs4791072, rs11079653, rs62069959, rs744214, rs7225452, rs6504424, |
| | rs973753, rs61687889, rs56399646, rs9635753, rs9914792, rs7220480, |
| | rs9914723, rs9903921, rs35105653, rs200369418, rs227912, |
| | rs58162388, rs9890911, rs11079656, rs12945884, rs7218425 |

PRS was calculated as a weighted sum of products between number of risk alleles and their corresponding regression coefficients. The full model included PRS and non-genetic predictors. A stepwise approach was exploited for selecting covariates for a reduced model. Covariables associated at p<0.05 entered a final predictive model. For model performances we used the area under the receiver operating characteristics curve (AUC). AUCs with 95% confidence intervals for clinical and genetic models were used for model comparison in SAS 9.4 (SAS. Cary, N.C.).

The polygenic risk scores (PRS) ranged from 12.1 to 35.7 (mean: 25.2; SD 4.4) and were normally distributed. The full multiple regression model inclusive of PRS is presented in Table 10. Two predictors were remained in the reduced final models after stepwise selection. Our final predictive model is presented in Table 10 (where CASI: Childhood anxiety sensitivity index; OR: Odds ratio; BS: Bootstrapping; AUC: Area under curve of pain scores over postoperative days 1 and 2 (POD12) after spine fusion; PRS: Polygenic risk score.)

TABLE 10
Multiple regression models evaluated for prediction of chronic post-surgical
pain (CPSP) and results of bootstrapping.

| Independent | Odds | | Upper | |
|---|---|---|---|---|
| Variable | Ratio | Lower 95% | 95% | P-values |

Full model

| | | | | |
|---|---|---|---|---|
| CASI | 1.416 | 1.129 | 1.774 | 0.0026 |
| Surgical Duration | 2.974 | 0.978 | 9.048 | 0.0548 |
| Pain_AUC_POD12 | 1.013 | 0.998 | 1.028 | 0.0892 |
| Weighted PRS | 2.462 | 1.473 | 4.117 | 0.0006 |

Reduced model

| | | | | |
|---|---|---|---|---|
| CASI | 1.313 | 1.085 | 1.501 | 0.005 |
| Weighted PRS | 2.117 | 1.501 | 2.985 | <0.001 |

After bootstrapping-final model

| | | mean OR | LCI from | UCI from BS |
|---|---|---|---|---|
| | Bias | from BS | BS for OR | for OR |
| Weighted PRS | 0.07 | 2.26 | 1.75 | 3.44 |
| CASI | 0.01 | 1.33 | 1.10 | 1.69 |

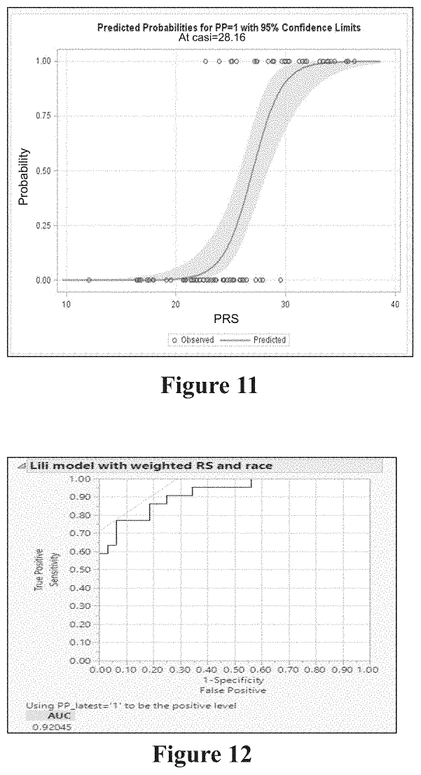

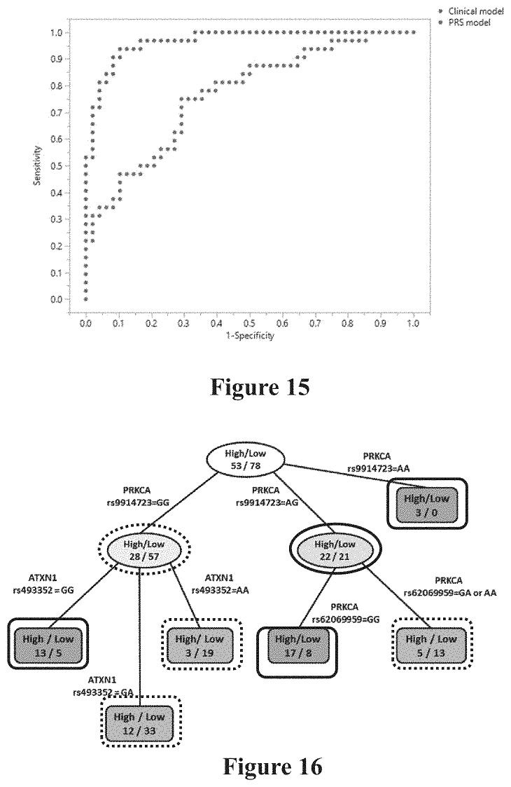

The predicted probability (with 95% CI) of CPSP for a subject having a median (for the cohort) CASI=28.16 using the regression model is plotted as a function of the PRS in FIG. 11. The probability of CPSP is higher than 50% at a PRS>26, Comparison of performance of the predictive model with three clinical predictors (CASI, surgery duration, and acute pain) and performance of the predictive model with generic predictor (PRS and CASI) showed statistically significant higher performance of genetic model. C-statistics for genetic model was 0.97 (95% CI 0.93-0.99) compared to 0.77 (95% CI 0.66-0.87) for non-genetic model (p=0.0007), FIG. 15.

Further, PRS predicted continuous AUC and dichotomous CPSP outcomes (p<0.0001). PRS were included in a multiple regression model with CASI, surgical duration to predict pain scores at 6-12 months after surgery. It remained a significant predictor for CPSP after adjusting for the other covariates (p<0.0001). As such, data show that Inclusion of PRS improved predictive accuracy for CPSP to 92% and explained 50% variability. FIG. 12.

### Example 5. Building a Predictive Model and Internal Validation

The predictive model for CSPS was calculated for the Scoliosis Cohort (described in Examples 1 and 2) based on significant psychophysical predictors (identified in Example 2) and PRS (identified in Example 4) using a bootstrap method. Briefly, bootstrapping was used to build and internally validate the prediction model. At each iteration, a random bootstrap sample the same size as the original sample was drawn with replacement from the original sample. Stepwise selection was used to derive a final model for each outcome where the final model will either include only variables with a p<0.05 or be the one minimizing marginal Akaike Information Criterion (AIC) and/or Bayes Information Criterion (BIC). Both criteria penalized larger models, although BIC model heavily, therefore balance fit with model size and avoid overfitting. The procedure was repeated for at least 100 times. To assess predictive performance, the models developed in each bootstrap sample were evaluated in both the bootstrap sample (apparent performance) and the original sample (test performance) using area under the receiver operating characteristic (ROC) curve (or the c statistics) and calibration slope for binary outcomes, and mean square error (MSE) and marginal, conditional, and fitted R² for continuous outcomes. Optimism was estimated to equal average (bootstrap performance−test performance) as internally validated performance as well as the variance based on cross-validation (VEcv). Specifically, with bootstrapping the model parameters (regression coefficients) and model performance (AUC) were evaluated. At each iteration (n=1000), a random bootstrap sample the same size as the original sample was drawn with replacement from the original sample. Logistic models were generated for each bootstrap sample and bootstrapping results were compared with results from the original model. Bootstrapping was performed in R software with the package boot. Mean OR and AUC for the ROC after bootstrapping and 95% CI are given in Table 10.

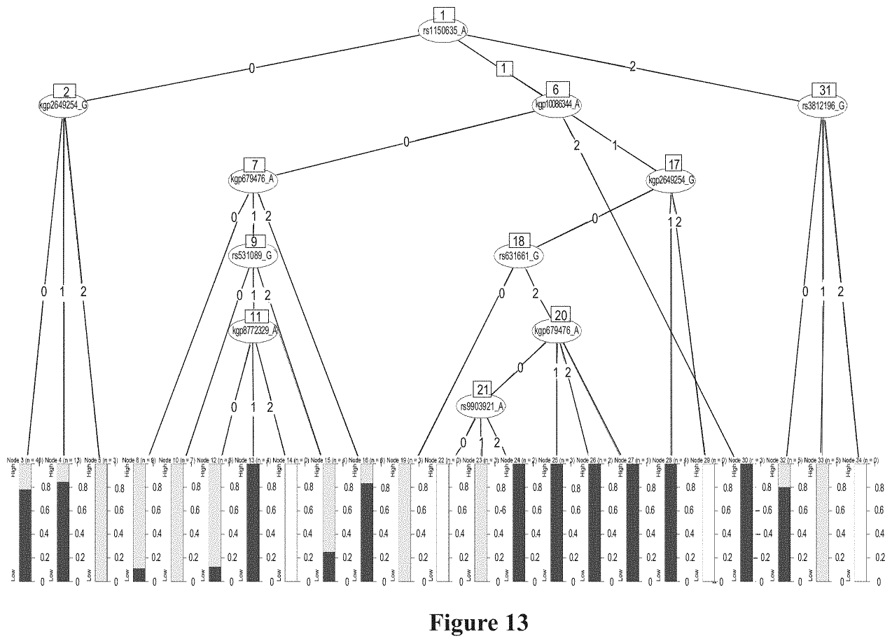

A decision tree was constructed from the top 80 most significant SNPs for CPSP (by chi-square) using C4.5 algorithm (J48 in R package RWeka) and partykit package for visualization. Generation of a decision tree can be used for predicting genetic signatures and to identify most important variant combinations that define risk strata. Here, the algorithm effectively classified subjects (correctly classified 87% and misclassified 13% subjects). FIG. 13.

Decision tree helped identify most informative SNP combinations (and the resulting strata), and to derive simple and easy to interpret logical rules, such as PRKCA rs9914723=AG AND PRKCA rs62069959=GG THEN Risk=High. FIG. 16. The training classification accuracy with the 3 SNPs in the figure (rs9914723, rs62069959 and rs493352) is 74% and can be further increased to 77% by adding two more SNPs (which are rs1150635 and rs1992701). In 5-fold cross validation the top three SNPs (and it especially true of the top rs9914723) are selected with a sufficient frequency to indicate that their selection as most discriminating features (also in combination) is quite robust. The actual confusion matrix for this tree is (low high, low high): 64 20, 14 33. As can be seen from the tree, just having at least one minor (risk) allele for rs9914723, increases the risk from 37% to about 54%.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A method of assessing risk of chronic post-surgical pain (CPSP) in a subject, the method comprising:
(i) obtaining a biological sample from a subject;
(ii) analyzing the biological sample to determine a genetic profile of the subject, wherein the genetic profile comprises a combination of single nucleotide polymorphisms (SNPs) in one or more of the genes selected from the group consisting of ATXN1, PRKCA, CACNG2, CTSG, DRD2, KCND2, KCNJ3, KCNJ6, and KCNK3;
(iii) calculating a polygenic risk score (PRS) based on the genetic profile determined in step (ii); and
(iv) assessing a risk of developing CPSP in the subject based on the PRS.

2. The method of claim 1, further comprising applying to the subject a pain management approach based on the subject's risk of developing CPSP.

3. The method of claim 1, wherein the combination of SNPs comprises SNPs selected from the following:
(a) rs61131185, rs12665284, rs493352, rs12198202, rs1150635, rs3812204, rs4716060, rs531089, and/or rs6459476 in ATXN1;
(b) rs9754467 in CACNG2;
(c) rs1957523 in CTSG;
(d) rs7125415 in DRD2;
(e) rs202146909 and/or rs1992701 in KCNJ3;
(f) rs2850125 in KCNJ6;
(g) rs2891519 in KCNK3;
(h) rs7809109 in KCND2; and
(i) rs62069959, rs11079653, rs9914723, rs7220480, rs200369418, rs227912, and/or rs744214 in PRKCA.

4. The method of claim 3, wherein the combination of SNPs comprises:
(a) rs61131185, rs12665284, rs493352, rs12198202, rs1150635, rs3812204, rs4716060, rs531089, and rs6459476 in ATXN1;
(b) rs9754467 in CACNG2;
(c) rs1957523 in CTSG;
(d) rs7125415 in DRD2;
(e) rs202146909 and rs1992701 in KCNJ3;
(f) rs2850125 in KCNJ6;
(g) rs2891519 in KCNK3;
(h) rs7809109 in KCND2; and
(i) rs62069959, rs11079653, rs9914723, rs7220480, rs200369418, rs227912, and rs744214 in PRKCA.

5. The method of claim 3, wherein the combination of SNPs consists of:
(a) rs61131185, rs12665284, rs493352, rs12198202, rs1150635, rs3812204, rs4716060, rs531089, and rs6459476 in ATXN1;
(b) rs9754467 in CACNG2;
(c) rs1957523 in CTSG;
(d) rs7125415 in DRD2;
(e) rs202146909 and rs1992701 in KCNJ3;
(f) rs2850125 in KCNJ6;
(g) rs2891519 in KCNK3;
(h) rs7809109 in KCND2; and
(i) rs62069959, rs11079653, rs9914723, rs7220480, rs200369418, rs227912, and rs744214 in PRKCA.

6. The method of claim 1, wherein the one or more genes are selected from the group consisting of CACNG2, DRD2, KCND2, KCNJ3, KCNJ6, and PRKCA.

7. The method of claim 6, wherein the combination of SNPs comprises SNPs selected from the following:
(a) rs9754467 and/or rs713952 in CACNG2;
(b) rs7125415 in DRD2;
(c) rs17376373, rs10488301, rs7809109, rs67881942, and/or rs17142908 in KCND2;
(d) rs202146909, rs77929576, and/or rs1992701 in KCNJ3;
(e) rs7281804, rs2836019, rs2850125, rs1892681, rs11088404, rs858018, rs13048511, rs7280905, and/or rs2032090 in KCNJ6; and
(f) rs4791072, rs11079653, rs62069959, rs744214, rs7225452, rs6504424, rs973753, rs61687889, rs56399646, rs9635753, rs9914792, rs7220480, rs9914723, rs9903921, rs35105653, rs200369418, rs227912, rs58162388, rs9890911, rs11079656, rs12945884, and/or rs7218425 in PRKCA.

8. The method of claim 7, wherein the combination of SNPs comprises:
(a) rs9754467 and rs713952 in CACNG2;
(b) rs7125415 in DRD2;
(c) rs17376373, rs10488301, rs7809109, rs67881942, and rs17142908 in KCND2;
(d) rs202146909, rs77929576, and rs1992701 in KCNJ3;
(e) rs7281804, rs2836019, rs2850125, rs1892681, rs11088404, rs858018, rs13048511, rs7280905, and rs2032090 in KCNJ6; and
(f) rs4791072, rs11079653, rs62069959, rs744214, rs7225452, rs6504424, rs973753, rs61687889, rs56399646, rs9635753, rs9914792, rs7220480, rs9914723, rs9903921, rs35105653, rs200369418, rs227912, rs58162388, rs9890911, rs11079656, rs12945884, and rs7218425 in PRKCA.

9. The method of claim 7, wherein the combination of SNPs consists of:
(a) rs9754467 and rs713952 in CACNG2;
(b) rs7125415 in DRD2;
(c) rs17376373, rs10488301, rs7809109, rs67881942, and rs17142908 in KCND2;
(d) rs202146909, rs77929576, and rs1992701 in KCNJ3;
(e) rs7281804, rs2836019, rs2850125, rs1892681, rs11088404, rs858018, rs13048511, rs7280905, and rs2032090 in KCNJ6; and
(f) rs4791072, rs11079653, rs62069959, rs744214, rs7225452, rs6504424, rs973753, rs61687889, rs56399646, rs9635753, rs9914792, rs7220480, rs9914723, rs9903921, rs35105653, rs200369418, rs227912, rs58162388, rs9890911, rs11079656, rs12945884, and rs7218425 in PRKCA.

10. The method of claim 1, wherein the subject is a human patient who is scheduled for or has undergone a surgery.

11. The method of claim 10, wherein the human patient is a child or an adolescent.

12. The method of claim 10, wherein the surgery is a spine and/or pectus surgery.

13. The method of claim 1, further comprising determining one or more non-genetic factors of the subject, wherein the one or more non-genetic factors are selected from the group consisting of childhood anxiety sensitivity index (CASI), pain catastrophizing (PCS-C) score, pediatric pain sensitivity score, surgical duration, and area under curve (AUC) of a pain score at days 1 and 2 post-surgery (Pain_AUC_POD12).

14. The method of claim 1, wherein the PRS is weighted by regression coefficients of the SNPs in the SNP combination to produce a weighted PRS.

15. The method of claim 14, wherein the risk of CPSP assessed in step (iv) is based on the weighted PRS in combination with one or more of the non-genetic factors by a regression model.

16. The method of claim 15, wherein the risk of CPSP assessed in step (iv) is based on:
(a) CASI, surgical duration, Pain_AUC_POD12, and weighted PRS; or
(b) CASI and weighted PRS.

17. The method of claim 2, wherein the pain management approach comprises administration of an analgesic to the subject before and/or after the surgery.

18. The method of claim 17, wherein the analgesic is locally or systemically administered.

19. The method of claim 17, wherein the analgesic comprises bupivacaine, ropivacaine, prilocaine, mepivacaine, chloroprocaine, lidocaine, gabapentin, morphine, fentanyl, oxycodone, hydrocodone, codeine, clonidine, or a combination thereof.

20. The method of claim 2, wherein the pain management approach comprises a psychosocial therapy.

21. A method for determining a genetic profile, the method comprising:
(a) obtaining a biological sample from a subject in need thereof,
(b) isolating nucleic acids from the biological sample,
(c) detecting a combination of single nucleotide polymorphisms (SNPs) in one or more of the genes selected from the group consisting of ATXN1, PRKCA, CACNG2, CTSG, DRD2, KCND2, KCNJ3, KCNJ6, and KCNK3; and
(d) determining a genetic profile of the subject based on the SNP combination detected in step (c).

22. The method of claim 21, wherein the combination of SNPs comprises SNPs selected from the following:
(a) rs61131185, rs12665284, rs493352, rs12198202, rs1150635, rs3812204, rs4716060, rs531089, and/or rs6459476 in ATXN1;
(b) rs9754467 in CACNG2;
(c) rs1957523 in CTSG;
(d) rs7125415 in DRD2;
(e) rs202146909 and/or rs1992701 in KCNJ3;
(f) rs2850125 in KCNJ6;
(g) rs2891519 in KCNK3;
(h) rs7809109 in KCND2; and
(i) rs62069959, rs11079653, rs9914723, rs7220480, rs200369418, rs227912, and/or rs744214 in PRKCA.

23. The method of claim 22, wherein the combination of SNPs comprises:
(a) rs61131185, rs12665284, rs493352, rs12198202, rs1150635, rs3812204, rs4716060, rs531089, and rs6459476 in ATXN1;
(b) rs9754467 in CACNG2;
(c) rs1957523 in CTSG;
(d) rs7125415 in DRD2;
(e) rs202146909 and rs1992701 in KCNJ3;
(f) rs2850125 in KCNJ6;
(g) rs2891519 in KCNK3;
(h) rs7809109 in KCND2; and
(i) rs62069959, rs11079653, rs9914723, rs7220480, rs200369418, rs227912, and rs744214 in PRKCA.

24. The method of claim 22, wherein the combination of SNPs consists of:
(a) rs61131185, rs12665284, rs493352, rs12198202, rs1150635, rs3812204, rs4716060, rs531089, and rs6459476 in ATXN1;
(b) rs9754467 in CACNG2;
(c) rs1957523 in CTSG;
(d) rs7125415 in DRD2;
(e) rs202146909 and rs1992701 in KCNJ3;
(f) rs2850125 in KCNJ6;
(g) rs2891519 in KCNK3;
(h) rs7809109 in KCND2; and
(i) rs62069959, rs11079653, rs9914723, rs7220480, rs200369418, rs227912, and rs744214 in PRKCA.

25. The method of claim 21, wherein the one or more genes are selected from the group consisting of CACNG2, DRD2, KCND2, KCNJ3, KCNJ6, and PRKCA.

26. The method of claim 25, wherein the combination of SNPs comprises SNPs selected from the following:
(a) rs9754467 and/or rs713952 in CACNG2;
(b) rs7125415 in DRD2;
(c) rs17376373, rs10488301, rs7809109, rs67881942, and/or rs17142908 in KCND2;
(d) rs202146909, rs77929576, and/or rs1992701 in KCNJ3;
(e) rs7281804, rs2836019, rs2850125, rs1892681, rs11088404, rs858018, rs13048511, rs7280905, and/or rs2032090 in KCNJ6; and
(f) rs4791072, rs11079653, rs62069959, rs744214, rs7225452, rs6504424, rs973753, rs61687889, rs56399646, rs9635753, rs9914792, rs7220480, rs9914723, rs9903921, rs35105653, rs200369418, rs227912, rs58162388, rs9890911, rs11079656, rs12945884, and/or rs7218425 in PRKCA.

27. The method of claim 26, wherein the combination of SNPs comprises:
(a) rs9754467 and rs713952 in CACNG2;
(b) rs7125415 in DRD2;
(c) rs17376373, rs10488301, rs7809109, rs67881942, and rs17142908 in KCND2;
(d) rs202146909, rs77929576, and rs1992701 in KCNJ3;
(e) rs7281804, rs2836019, rs2850125, rs1892681, rs11088404, rs858018, rs13048511, rs7280905, and rs2032090 in KCNJ6; and
(f) rs4791072, rs11079653, rs62069959, rs744214, rs7225452, rs6504424, rs973753, rs61687889, rs56399646, rs9635753, rs9914792, rs7220480, rs9914723, rs9903921, rs35105653, rs200369418, rs227912, rs58162388, rs9890911, rs11079656, rs12945884, and rs7218425 in PRKCA.

28. The method of claim 26, wherein the combination of SNPs consists of:
(a) rs9754467 and rs713952 in CACNG2;
(b) rs7125415 in DRD2;
(c) rs17376373, rs10488301, rs7809109, rs67881942, and rs17142908 in KCND2;
(d) rs202146909, rs77929576, and rs1992701 in KCNJ3;
(e) rs7281804, rs2836019, rs2850125, rs1892681, rs11088404, rs858018, rs13048511, rs7280905, and rs2032090 in KCNJ6; and
(f) rs4791072, rs11079653, rs62069959, rs744214, rs7225452, rs6504424, rs973753, rs61687889, rs56399646, rs9635753, rs9914792, rs7220480, rs9914723, rs9903921, rs35105653, rs200369418, rs227912, rs58162388, rs9890911, rs11079656, rs12945884, and rs7218425 in PRKCA.

29. The method of claim 21, wherein the subject in need thereof is a human patient who is scheduled for or has undergone a surgery.

30. The method of claim 29, wherein the human patient is a child or an adolescent.

31. The method of claim 29, wherein the surgery is a spine and/or pectus surgery.

32. The method of claim 21, wherein the biological sample from a subject in need thereof is blood, plasma, salvia, urine, sweat, feces, a buccal smear, or a tissue.

33. A kit for determining a genetic portfolio of a subject, the kit comprising: (i) means for determining a combination of single nucleotide polymorphisms (SNPs) in one or more of the genes selected from the group consisting of ATXN1, PRKCA, CACNG2, CTSG, DRD2, KCND2, KCNJ3, KCNJ6, and KCNK3.

34. The kit of claim 33, wherein the combination of SNPs comprises SNPs selected from the following:
(a) rs61131185, rs12665284, rs493352, rs12198202, rs1150635, rs3812204, rs4716060, rs531089, and/or rs6459476 in ATXN1;
(b) rs9754467 in CACNG2;
(c) rs1957523 in CTSG;
(d) rs7125415 in DRD2;
(e) rs202146909 and/or rs1992701 in KCNJ3;
(f) rs2850125 in KCNJ6;
(g) rs2891519 in KCNK3;
(h) rs7809109 in KCND2; and
(i) rs62069959, rs11079653, rs9914723, rs7220480, rs200369418, rs227912, and/or rs744214 in PRKCA.

35. The kit of claim 34, wherein the combination of SNPs comprises:
(a) rs61131185, rs12665284, rs493352, rs12198202, rs1150635, rs3812204, rs4716060, rs531089, and rs6459476 in ATXN1;
(b) rs9754467 in CACNG2;
(c) rs1957523 in CTSG;
(d) rs7125415 in DRD2;
(e) rs202146909 and rs1992701 in KCNJ3;
(f) rs2850125 in KCNJ6;
(g) rs2891519 in KCNK3;
(h) rs7809109 in KCND2; and
(i) rs62069959, rs11079653, rs9914723, rs7220480, rs200369418, rs227912, and rs744214 in PRKCA.

36. The kit of claim 34, wherein the combination of SNPs consists of:
(a) rs61131185, rs12665284, rs493352, rs12198202, rs1150635, rs3812204, rs4716060, rs531089, and rs6459476 in ATXN1;
(b) rs9754467 in CACNG2;
(c) rs1957523 in CTSG;
(d) rs7125415 in DRD2;
(e) rs202146909 and rs1992701 in KCNJ3;
(f) rs2850125 in KCNJ6;
(g) rs2891519 in KCNK3;
(h) rs7809109 in KCND2; and
(i) rs62069959, rs11079653, rs9914723, rs7220480, rs200369418, rs227912, and rs744214 in PRKCA.

37. The kit of claim 33, wherein the one or more genes are selected from the group consisting of CACNG2, DRD2, KCND2, KCNJ3, KCNJ6, and PRKCA.

38. The kit of claim 37, wherein the combination of SNPs comprises SNPs selected from the following:
(a) rs9754467 and/or rs713952 in CACNG2;
(b) rs7125415 in DRD2;
(c) rs17376373, rs10488301, rs7809109, rs67881942, and/or rs17142908 in KCND2;
(d) rs202146909, rs77929576, and/or rs1992701 in KCNJ3;
(e) rs7281804, rs2836019, rs2850125, rs1892681, rs11088404, rs858018, rs13048511, rs7280905, and/or rs2032090 in KCNJ6; and
(f) rs4791072, rs11079653, rs62069959, rs744214, rs7225452, rs6504424, rs973753, rs61687889, rs56399646, rs9635753, rs9914792, rs7220480, rs9914723, rs9903921, rs35105653, rs200369418, rs227912, rs58162388, rs9890911, rs11079656, rs12945884, and/or rs7218425 in PRKCA.

39. The kit of claim 38, wherein the combination of SNPs comprises:
(a) rs9754467 and rs713952 in CACNG2;
(b) rs7125415 in DRD2;
(c) rs17376373, rs10488301, rs7809109, rs67881942, and rs17142908 in KCND2;
(d) rs202146909, rs77929576, and rs1992701 in KCNJ3;
(e) rs7281804, rs2836019, rs2850125, rs1892681, rs11088404, rs858018, rs13048511, rs7280905, and rs2032090 in KCNJ6; and
(f) rs4791072, rs11079653, rs62069959, rs744214, rs7225452, rs6504424, rs973753, rs61687889, rs56399646, rs9635753, rs9914792, rs7220480, rs9914723, rs9903921, rs35105653, rs200369418, rs227912, rs58162388, rs9890911, rs11079656, rs12945884, and rs7218425 in PRKCA.

40. The kit of claim 38, wherein the combination of SNPs consists of:
(a) rs9754467 and rs713952 in CACNG2;
(b) rs7125415 in DRD2;
(c) rs17376373, rs10488301, rs7809109, rs67881942, and rs17142908 in KCND2;
(d) rs202146909, rs77929576, and rs1992701 in KCNJ3;
(e) rs7281804, rs2836019, rs2850125, rs1892681, rs11088404, rs858018, rs13048511, rs7280905, and rs2032090 in KCNJ6; and
(f) rs4791072, rs11079653, rs62069959, rs744214, rs7225452, rs6504424, rs973753, rs61687889, rs56399646, rs9635753, rs9914792, rs7220480, rs9914723, rs9903921, rs35105653, rs200369418, rs227912, rs58162388, rs9890911, rs11079656, rs12945884, and rs7218425 in PRKCA.

41. The kit of claim 33, wherein the means for determining the combination of SNPs comprise a set of primer pairs, each of which is for amplifying a fragment encompassing one of the SNPs in the combination and wherein the set of primer pairs collectively amply fragments encompassing the SNPs in the combination.

42. The kit of claim 33, wherein the means for determining the combination of SNPs comprise a set of oligonucleotides, each of which is for detecting one of the SNPs in the combination, and wherein the set of oligonucleotides collectively detects the SNPs in the combination.

43. The kit of claim 42, wherein the set of oligonucleotides is attached to a microarray chip.

44. The kit of claim 33, wherein the kit further comprises:
(ii) a tool for collecting a biological sample from a subject;
(iii) a container for placing the biological sample; and/or
(iv) one or more reagents for extracting nucleic acids from the biological sample.

45. A method of assessing risk of chronic post-surgical pain (CPSP) in a subject, the method comprising:
(i) determining one or more non-genetic factors of a subject, wherein the one or more non-genetic factors are selected from the group consisting of childhood anxiety sensitivity index (CASI), pain catastrophizing (PCS-C) score, pediatric pain sensitivity score, surgical duration, and area under curve (AUC) of a pain score at days 1 and 2 post-surgery (Pain_AUC_POD12); and
(ii) assessing risk of CPSP of the subject based on the one or more non-genetic factors.

46. The method of claim 45, further comprising applying to the subject a pain management approach based on the subject's risk of developing CPSP.

47. The method of claim 45, wherein the subject is a human patient who is scheduled for or has undergone a surgery.

48. The method of claim 47, wherein the human patient is a child or an adolescent.

49. The method of claim 47, wherein the surgery is a spine and/or pectus surgery.

50. The method of claim 47, wherein the pain management approach comprises administration of an analgesic to the subject before and/or after the surgery.

51. The method of claim 50, wherein the analgesic is locally or systemically administered.

52. The method of claim 50, wherein the analgesic comprises bupivacaine, ropivacaine, prilocaine, mepivacaine, chloroprocaine, lidocaine, gabapentin, morphine, fentanyl, oxycodone, hydrocodone, codeine, clonidine, or a combination thereof.

53. The method of claim 47, wherein the pain management approach comprises a psychosocial therapy.
